SEQUENCE LISTING (108 sequences)

SEQ ID NO: 1 - protein, 355 aa, Arabidopsis thaliana
MNIVSWKDANDEVAGGATTRREREVKEDQEETEVRATSGKTVIKKQPTSISSSSSSWMKSKDPRIVRVSRAFGGKDRHSKVCTLRGLRDRRVRLSVPTAIQLYDLQERLGVDQPSKAVDWLLDAAKEEIDELPPLPISPENFSIFNHHQSFLNLGQRPGQDPTQLGFKINGCVQKSTTTSREENDREKGENDVVYTNNHHVGSYGTYHNLEHHHHHHQHLSLQADYHSHQLHSLVPFPSQILVCPMTTSPTTTTIQSLFPSSSSAGSGTMETLDPRQMVSHFQMPLMGNSSSSSSQNISTLYSLLHGSSSNNGGRDIDNRMSSVQFNRTNSTTTANMSRHLGSERCTSRGSDHHM

SEQ ID NO: 2 - DNA, 1068 bp, Arabidopsis thaliana
atgaatatcgtctcttggaaagatgcaaacgacgaagttgcaggcggcgctacgacaagacgtgaaagagaagtaaaagaggatcaagaagaaaccgaagtcagagccaccagtggcaaaaccgtaattaaaaagcagcctacatcgatctcttcttcttcttcttcgtggatgaaatccaaggatccgaggattgttagggtttcacgcgcctttggaggcaaagaccgtcacagcaaagtgtgtacgttacgtggactacgtgacagacgcgtgagattatcagtcccaacggctattcagctctacgatcttcaagaacggctcggtgttgaccagcctagcaaagccgttgactggttgcttgatgcagctaaagaggagatcgacgagctacctccgttacctatctcgccggaaaatttcagcatcttcaaccatcatcagtccttcttgaatcttggtcaacggcccggtcaagatccgacccaactcgggtttaaaatcaatggatgtgtacaaaagtctactactactagccgcgaagaaaacgatagagagaaaggagaaaacgatgtcgtttacacaaacaatcatcatgttgggtcttatggaacttatcacaacctggaacatcatcatcatcatcaccaacatttgagtttacaggcagattatcatagtcatcaactacatagtcttgtcccatttccatcacaaattttggtatgtccaatgacgacatcaccaacaactacaactatacaatctttgtttccatcatcatcgtcagctggttcagggactatggagacattagatccgaggcaaatggtaagccattttcaaatgccattaatgggtaattcttcatcttcgtcttcccaaaacatttcgacattatattcgttgttacatggtagtagtagcaacaatggtggtagagacattgataatcggatgtcgtcggtccaattcaaccgaactaatagcactacaacggctaacatgtcgaggcatctaggctcggagcgttgtacaagtagaggaagtgatcaccatatgtga

SEQ ID NO: 3 - protein, 278 aa, Arabidopsis thaliana
MNIVSWKDANDEVAGGATTRREREVKEDQEETEVRATSGKTVIKKQPTSISSSSSSWMKSKDPRIVRVSRAFGGKDRHSKVCTLRGLRDRRVRLSVPTAIQLYDLQERLGVDQPSKAVDWLLDAAKEEIDELPPLPISPENFSIFNHHQSFLNLGQRPGQDPTQLGFKINGCVQKSTTTSREENDREKGENDVVYTNNHHVGSYGTYHNLEHHHHHHQHLSLQADYHSHQLHSLVPFPSQILVCPMTTSPTTTTIQSLFPSSSSAGSGTMETLDPRQM

SEQ ID NO: 4 - DNA, 837 bp, Arabidopsis thaliana
atgaatatcgtctcttggaaagatgcaaacgacgaagttgcaggcggcgctacgacaagacgtgaaagagaagtaaaagaggatcaagaagaaaccgaagtcagagccaccagtggcaaaaccgtaattaaaaagcagcctacatcgatctcttcttcttcttcttcgtggatgaaatccaaggatccgaggattgttagggtttcacgcgcctttggaggcaaagaccgtcacagcaaagtgtgtacgttacgtggactacgtgacagacgcgtgagattatcagtcccaacggctattcagctctacgatcttcaagaacggctcggtgttgaccagcctagcaaagccgttgactggttgcttgatgcagctaaagaggagatcgacgagctacctccgttacctatctcgccggaaaatttcagcatcttcaaccatcatcagtccttcttgaatcttggtcaacggcccggtcaagatccgacccaactcgggtttaaaatcaatggatgtgtacaaaagtctactactactagccgcgaagaaaacgatagagagaaaggagaaaacgatgtcgtttacacaaacaatcatcatgttgggtcttatggaacttatcacaacctggaacatcatcatcatcatcaccaacatttgagtttacaggcagattatcatagtcatcaactacatagtcttgtcccatttccatcacaaattttggtatgtccaatgacgacatcaccaacaactacaactatacaatctttgtttccatcatcatcgtcagctggttcagggactatggagacattagatccgaggcaaatgtag

SEQ ID NO: 5 - protein, 360 aa, Arabidopsis thaliana
MRSGECDEEEIQAKQERDQNQNHQVNLNHMLQQQQPSSVSSSRQWTSAFRNPRIVRVSRTFGGKDRHSKVCTVRGLRDRRIRLSVPTAIQLYDLQDRLGLSQPSKVIDWLLEAAKDDVDKLPPLQFPHGFNQMYPNLIFGNSGFGESPSSTTSTTFPGTNLGFLENWDLGGSSRTRARLTDTTTTQRESFDLDKGKWIKNDENSNQDHQGFNTNHQQQFPLTNPYNNTSAYYNLGHLQQSLDQSGNNVTVAISNVAANNNNNLNLHPPSSSAGDGSQLFFGPTPPAMSSLFPTYPSFLGASHHHHVVDGAGHLQLFSSNSNTASQQHMMPGNTSLIRPFHHLMSSNHDTDHHSSDNESDS

SEQ ID NO: 6 - DNA, 1083 bp, Arabidopsis thaliana
atgagatcaggagaatgtgatgaagaggagattcaagcaaagcaagaaagagatcaaaatcaaaatcatcaagtaaacttaaaccacatgttgcaacaacaacagccgagttcggtatcatcttcaaggcaatggacttcagcttttaggaatccaagaatcgttcgagtctcaagaacattcggtggcaaagacagacacagcaaagtatgtacagtccgtggtcttcgagaccggaggataaggttgtccgtacctacagctattcaactctacgaccttcaagatcgattagggctgagtcagccaagcaaagtcattgattggttactcgaagcagcaaaagatgacgtagacaagctacctcctctacaattcccacatggatttaaccagatgtatccaaatctcatcttcggaaactccgggtttggagaatctccatcttcaactacatcaacaacgtttccaggaaccaatctcgggttcttggaaaattgggatcttggtggttcttcaagaacaagagcaagattaaccgatacaactacgacccaaagagaaagttttgatcttgataaaggaaaatggatcaaaaacgacgagaatagtaatcaagatcatcaagggtttaacaccaatcatcaacaacaatttcctctgaccaatccgtacaacaacacttcagcttattacaaccttggacatcttcaacaatcgttagaccaatctggtaataacgttactgtcgcaatatctaatgttgctgctaataataacaataatctcaatttgcatcctccttcctcgtctgccggagatggatctcagctttttttcggtcctactcctccggcaatgagctctctattcccgacatacccttcgtttcttggagcttctcatcatcatcatgtcgtcgatggagccggtcatcttcagctctttagctcgaattcaaataccgcatcgcagcaacacatgatgccgggtaatacgagtttgattagaccatttcatcatttgatgagctcgaatcatgatacggatcatcatagtagcgataatgaatcagattcttga

SEQ ID NO: 7 - protein, 242 aa, Arabidopsis thaliana
MGIKKEDQKSSLSLLTQRWNNPRIVRVSRAFGGKDRHSKVCTVRGLRDRRIRLSVMTAIQVYDLQERLGLSQPSKVIDWLLEVAKNDVDLLPPLQFPPGFHQLNPNLTGLGESFPGVFDLGRTQREALDLEKRKWVNLDHVFDHIDHHNHFSNSIQSNKLYFPTITSSSSSYHYNLGHLQQSLLDQSGNVTVAFSNNYNNNNLNPPAAETMSSLFPTRYPSFLGGGQLQLFSSTSSQPDHIE

SEQ ID NO: 8 - DNA, 729 bp, Arabidopsis thaliana
atgggaataaaaaaagaagatcagaaaagttccttgtccttgttgacgcaacgatggaataatccgaggatcgtgcgagtctcccgagcatttggaggcaaagatagacacagcaaagtgtgcacagttcgtggtctccgagacaggaggataaggttgtcggtgatgacagcgatccaagtctacgaccttcaagaacgattagggctgagtcagcctagcaaagtcattgattggctcttagaagttgccaaaaacgacgtcgatttgcttcctcctttgcaattcccacctggttttcaccaacttaaccctaatctcaccggccttggagaatcttttcccggggtcttcgatcttggaagaacacagagagaagctttggatcttgagaaaaggaaatgggttaatcttgatcatgtgtttgatcacatcgatcatcacaaccatttctcgaattcaatccagagcaacaagctttatttccctacaattacttcgtctagcagctcttatcactacaaccttggacatctccaacagtcactactagaccaatctggtaacgtcactgtcgcattttctaataactataataataataatctcaatcctccggcggcggaaactatgagttctctattcccaacaaggtacccttcgtttctcggtggtggtcaacttcaactctttagtagcacaagctcacaaccagaccatatcgagtag

SEQ ID NO: 9 - protein, 294 aa, Brachypodium distachyon
MMSGNQQQQSSSAVASAASEQEQQQQQAELARKHAAAVATSRQWSAQTESRIVRVSRVFGGKDRHSKVKTVKGLRDRRVRLSVPTAIQLYDLQDRLGLNQPSKVVDWLLNAARHEIDKLPPLQFPPHHGQQLNGADLMEHHMASASMLPLMPHDFASALAADHGGKGHAHDDVADMGRFAAAGYHRFMGSNSSPLGLVNGGMPYNFTGESWNNNSTGGAHENCQGSPHGGQVSIAAAHHSAFPSLLSLAPRSHQLVYYASEAAEQFPVDNNLGSQGLSLSSARAFHDHDHAAGS

SEQ ID NO: 10 - DNA, 885 bp, Brachypodium distachyon
atgatgagcggcaaccagcagcagcaatcgtcgtcggcggtggcgtcggccgcgtcggagcaggagcagcagcagcagcaggcggagctcgcccgcaagcacgcggcggcggtggcgacgtcgcggcaatggtcggcgcagacggagtcgcggatcgtgcgcgtgtcccgggtcttcgggggcaaggaccggcacagcaaggtgaagactgtgaaggggctccgcgacaggcgcgtgcggctgtccgtgcccacggccatccagctctacgacctgcaggaccgcctgggcctcaaccagcccagcaaggtcgtcgactggctcctcaacgctgcccgccacgagatcgacaagctccccccgctccagttcccgccccaccacggccagcagctcaacggcgccgacctcatggagcaccacatggcctctgcctccatgctgcctctcatgccgcacgacttcgcgtccgcgctcgccgctgatcatggcggcaagggacacgcccacgacgacgtggccgacatggggcgctttgccgccgctgggtaccaccggttcatggggagcaactcgtcgccgctgggactggtcaacggcgggatgccgtacaacttcaccggtgagtcatggaataataacagcactggtggtgcacacgagaactgccaagggtcgccccatggaggccaggtctccatcgcggcagcccaccactcggcgttcccgtcgttgctctccttggctccgaggtcgcaccaactcgtctactacgcgtcggaggccgccgagcagttcccggttgataataacctgggctcgcaaggcttgtccttgagctccgcgagggctttccatgaccatgatcacgcagctggaagctag

SEQ ID NO: 11 - protein, 298 aa, Brachypodium distachyon
MHTNEEPGPDGAANKALLQLHQAASSAASSSSSRQWSAESRIVRVSRVFGGKDRHSKVRTVKGLRDRRVRLSVPTAIQLYDLQDRLGLSQPSKVVDWLLDAARREIDKLPPLNFPPPDPHAHLMAMAPPPFSATTAAPMLVDGDKAAGQLGRCDDGDELKGFMGLRSSLGLVSGAMTPSALAAHASPYHHQYGAGAEAWSNDAHDYLSGGGRGHNGTDSIQQVATAHGHGHSPYFPSLLSLAPAMSQFVFYSAAEGGFGSAMKEAGDGDDDDQFPVDNLDASQGQLSLSSDRSFLHSG

SEQ ID NO: 12 - DNA, 897 bp, Brachypodium distachyon
atgcacacaaacgaggagccgggcccggacggcgccgccaataaggcgctgctgcagctgcatcaggcggcgagctctgcagcctcgtcgtcgtcgtcgcggcagtggtcggcggagtcgcggatcgtgcgcgtgtcgcgggtgttcgggggcaaggaccggcacagcaaggtgcggaccgtgaaggggctccgggaccggcgtgtccggctgtccgtgcccacggccatccagctctacgacctccaggaccgcctcggcctctcccagcccagcaaggtcgtcgactggctcctcgacgccgcccgccgcgagatcgacaagctcccgcccctcaacttcccaccacccgatccccacgcgcacctcatggccatggcgccgccgccattctccgccacgacggcggcgcccatgctggtcgacggcgacaaggcggcgggccagctgggccgctgtgacgacggtgacgagctcaaggggttcatgggcctgaggagctcgctcggccttgtcagcggcgccatgacgccgtccgcgttggcggcccatgccagcccgtatcaccaccagtacggcgccggcgctgaggcttggagcaatgatgcgcacgattacctgagcggcggtggccgcggccataatggcacggattcgattcaacaggtggcgacggctcacgggcacggccactcgccgtattttccctcgctgctgtccttggcgccggcgatgtcacagttcgtgttctactcggccgcagagggcggcttcggatcagccatgaaagaggccggcgacggtgacgacgatgatcagttccccgtagataacctcgacgcttcacaagggcagctatcgctcagctccgacagaagcttccttcactccggttga

SEQ ID NO: 13 - protein, 185 aa, Brachypodium distachyon
MEEVGKENSNSSCKRLRGATGGSGGDSSAVAWRTSRVARAAAGGKDRHSKVVTSRGLRDRRIRLSVPTAIQFYDIQDRLGVDQPSKAIEWLIGAAASAIDALPSLDCSFALPAAASPPAAADDAESCLQLCLVEYHDPSAEANPGQKNMPATEDVGAMKTRRKQTHHLNFFHLLSARFSSAIVFL

SEQ ID NO: 14 - DNA, 558 bp, Brachypodium distachyon
atggaggaggtaggcaaggagaacagcaactcctcctgcaagcgcctgcgcggagcaacaggcggcagcggtggcgactcgtcggcggtggcatggaggacgtcccgggtggcgcgtgcggctgccggtggcaaggacaggcacagcaaggtggtgacctcacgcggcctccgggaccgccgcatccggctctccgtgccgacagcgatccaattctacgacatccaggaccgcctcggcgtcgaccagcccagcaaggccatcgagtggctcatcggcgccgccgcctccgccattgacgctctaccgtcgctggactgctcctttgccctccctgccgctgcttccccgccggcagctgctgatgatgctgaaagttgcctgcaactttgtttggtggagtatcatgatccatcagcagaagcaaatccaggacagaagaacatgccagccactgaagatgttggtgccatgaagacgcgtcgaaaacagacccatcacttgaattttttccatcttttatcagcgcgattttcatctgccatcgtttttctctaa

SEQ ID NO: 15 - protein, 382 aa, Brachypodium distachyon
MAAAGGQGAAAGGRRGGGGGGGGKRGRGERESGGATASASRICRGRANGGKDRHSKVVTSKGVRDRRVRLAVPTAIQFYDLQDRLGFDQPSKAVDWLLDAASHAIAKLPPLDPTAFSFPFPADDAQQATKKGKKPLLLQQGSSSTTSESDLSLSRSDAARDKQHKGAVVTVAAAPGSAQAASSFTELLSGNAIAAAEHIQSWYRQQQRQLSAAGPVGFAAHPGNNNRGASQDAACAGLRFGNAPPLGMAPARPLLPVRPVGFPTANSTGEIAPFSFVQGVLAAASAQAPAPAGDYSLDFSMSSGFTGGGVGSNSRGTLQSNSRSQYFSNQLQLYELDGPSPPFLYDPAAALAAHPSSENRHLTGTPPLQLWNGFRMEEKSQN

SEQ ID NO: 16 - DNA, 1149 bp, Brachypodium distachyon
atggctgcagctgggggccagggcgcggcggcggggggccggcgcggcggcggcggaggaggaggaggcaagcgcgggcgcggcgagagggagagcggtggcgcgacggcgtcggcgtcgcggatctgccgcgggagggcgaacggcggcaaggaccggcacagcaaggtggtgacgtcgaagggggtccgggaccggcgggtgcggctggcggtgcccacggccatccagttctacgacctgcaggaccgcctcggcttcgaccagcccagcaaggccgtcgactggctcctcgacgccgcctcccacgccatcgccaagctcccgcccctcgaccccaccgccttctccttccctttccccgccgacgacgcccagcaggccaccaagaaggggaagaagccgctgctgctccagcagggcagcagcagcaccacctcagagtccgacctctcgctctcacgctccgacgccgcccgcgacaagcagcacaagggggccgtcgtcaccgtcgccgctgcccccggatccgcgcaggccgcgtcctccttcaccgagctgctcagcggcaacgccattgccgccgccgagcacatccaatcctggtaccgacagcagcagcggcagctttccgccgcgggccccgtcggattcgcggcgcaccctgggaacaacaacagaggagcctcgcaagacgcggcgtgcgccggcctcagattcggcaatgcgcctccgctcggcatggcgcccgcgcggccgctcctgcccgtgcggccggtcggcttccccaccgccaattccacgggcgagatcgcgcccttctcgttcgtccagggcgtgctggctgcggcatccgctcaggccccggccccggccggagactacagcctcgacttctccatgtcgtcgggtttcacgggcggcggcgtcggcagcaacagcagggggacccttcagtccaactccaggtcacagtacttttcaaaccagctccagctctatgagctcgacggcccgagccctcccttcttgtatgatccagccgctgccctggccgcgcacccctcgtcggagaaccgccacctcaccggaacgccgccattgcagctctggaacgggttccggatggaggagaagagccagaactga

SEQ ID NO: 17 - protein, 442 aa, Brachypodium distachyon
MDLGGDVPLRKRGRNPEDDDGEETEQGGAKQQRRMGAVVPWPAQPPPQQQNPASRIYRVSRGSGGKDRHSKVYTAKGIRDRRVRLSVATAIQFYDLQDRLGYDQPSKAVEWLIKAAAAAIDKLPSLDASAFPNHPASASAAHNNKPHADASQQQLPRSGCSSTSETSKGSVLSLSRSDSRVKARDRSAKDKDDSALAAAGTTRRATAAAAATSSAQAASFTELLTGLAVASAAAGTTTTATTATAPVVAEHKPPPQQHNSWQPMPATADYLGFAQPRKPAHGMLHTTFASPAPHLANIAPIAMAATQHFSLTSGEPQQAEMAPFSFLQDHFMPVHAAAAAGPAAVDYNLNFSMSSGLVGVNSRGTLQSNSQPHLSSHHHHHHHQQQLQRLSAPLDASHIPFFFSPAVTVASPNPAEGHFGSAAAFQLWDGFRHSDVKEKGKN

SEQ ID NO: 18 - DNA, 1329 bp, Brachypodium distachyon
atggacctcggcggcgacgtgccgctacgcaagcggggacgaaacccggaggacgacgacggcgaggaaacggagcaagggggcgcgaagcagcagaggaggatgggcgcggtggtgccgtggccggcgcagccgccaccgcagcagcagaacccggcgtcgcggatctaccgcgtgtcgaggggctccggcgggaaggaccggcacagcaaggtgtacacggccaagggcatccgtgaccgccgcgtgcgcctctccgtcgccacagccatccagttctacgacctgcaggaccgcctcggctacgaccagcccagcaaggccgtcgagtggctcatcaaggccgccgccgccgccatcgacaagctgccctcactcgacgcatccgccttccccaaccaccccgcttccgcctccgctgcacacaacaacaagcctcacgccgatgcctcccagcagcagctcccgagatcgggctgcagcagcacctccgagaccagcaaaggctccgtcctctccctctcccgctccgacagccgcgtcaaggcccgcgaccgcagcgccaaagacaaggacgactccgctctagcagcagcagggacgacgcgacgcgccaccgccgccgccgccgcgacctcctcggcgcaggcggcttccttcacggagctgctcaccggactggcggtggccagcgccgccgccggtactactactactgctactaccgccaccgcgccggtcgttgccgagcacaagccgccgccgcagcagcacaactcctggcagccgatgcccgccaccgccgactacctcggcttcgcgcagccccggaagcccgcccacgggatgctgcacacaaccttcgcctccccggccccgcacctcgccaacattgcgcccatcgccatggcggccacgcaacacttcagcttgaccagtggcgagccgcagcaagccgagatggcgcccttctccttcttacaggaccacttcatgcccgtccacgcggcggcggcggcgggtccggcggccgtagactacaacctcaacttctccatgtcctcgggtctcgtgggtgtcaacagcagggggacccttcagtccaattcgcagccgcacctctccagccaccatcaccaccaccaccaccagcagcagctccagaggctgtctgctccactggatgcttcacacatacccttcttcttcagcccggccgtcaccgtggcctcgcccaacccggcggagggccacttcggctcggcggccgcgtttcagctctgggatgggttccggcactcggacgtcaaggagaagggcaagaactga

SEQ ID NO: 19 - protein, 296 aa, Glycine max
MIKSQKEADFQLKQEGLSQSINDPEKAKATSSSVAQWPRLKDPRIVRVSRAFGGKDRHSKVCTIRGLRDRRVRLSVPTAIQLYDLQDRLGLSQPSKVVDWLLNAAKHEIDELPPLPIIPSVNNFTLGYPSAVTSNEATTSNSQPNEQLLNINRSIQWEDSNHNSTWKLKAKEVSGEMRISKEAIIKDLAHIANLNHPSFLGLLNTMPHGYRWEHSAGDVSHPSNATIFPFTCYSNGHGSEANQPLSDVEFRLREASSITRYSTLPRFFQFIYSTKRSNMNGFLRVFCKNYPLSDNE

SEQ ID NO: 20 - DNA, 891 bp, Glycine max
atgattaagagccaaaaagaagcagattttcaattaaaacaggagggtctttctcaatcaattaatgatcctgaaaaggcaaaggctacatcaagctcagtagcacaatggccaagattgaaggatccaaggattgttcgagtatccagagcctttggaggaaaagacaggcatagcaaggtttgcactataagagggttaagagacagacgggtgaggctatcagtaccaactgccatccaactctatgatcttcaagataggctagggctcagtcaacctagcaaggttgttgattggttgctcaatgcagctaagcatgaaattgatgaacttccaccacttcccattattccttcagtgaataacttcacccttggttatccatctgctgttacttctaatgaagctaccacctcaaattctcagcctaatgagcaacttcttaatatcaacagaagcattcaatgggaagattcaaaccataattctacatggaaattaaaggctaaagaagtttcaggagaaatgaggataagcaaggaagcaataatcaaggacctagcacacatagcaaatcttaatcatccttcattcttgggcttgctgaatactatgccacatggttaccgatgggaacattctgctggtgatgtttctcatccgagcaacgcaaccatatttcccttcacatgttacagcaatggacatggatccgaggcaaatcaaccactatcagatgttgagttcagactacgagaagcttcttcaattacaagatattccacattaccacggttttttcagtttatatattccacgaaacgatctaacatgaatggctttctaagggtattttgtaagaactatcctctaagtgacaatgaatag

SEQ ID NO: 21 - protein, 290 aa, Glycine max
MINSTKETDFPLKQEGLSSSDHEKAKAPSSSSSSQWLKLKDPRIVRVSRAFGGKDRHSKVCTIRGLRDRRVRLSVPTAIHLYDLQDRLGLNQPSKVVDWLLNAAKHEIDELPPLPIPPGNFTLGYPSLVSCNEVSTSREEEKEMMMMIIIIIITKPSFFFGFDEHYAITCIDVVPFPSTLALSTGNSTSTTTTSQILVCPPPPGATTQPYFPSSHFATMEMNARQINHYQMLSSSSHQNLLANSLNHPSSQQLMSQSGKAPFSLRIRPKLFHSPNSSESHSQKDQDFPSK

SEQ ID NO: 22 - DNA, 873 bp, Glycine max
cacaatggctaaaactgaaggatccaaggattgttcgagtatcaagagcctttggaggaaaagacaggcatagcaaggtatgcacaataagaggactaagagatagaagagtgaggctttcagtccctacagcaattcacctctatgatctccaagataggcttggactcaaccaaccaagcaaggttgttgattggttgctcaatgcagcaaaacatgaaattgatgaattgccaccacttccaattcctccaggtaacttcacccttggttatccctctcttgtttcttgtaatgaagttagcacctctcgtgaaggttcaggccaaaacactttattatggaaaccaaagccaggagaaataatggttagtgatcatgataaggcaaattggatgaatagaagagaaggagatgaaataatctcttgccaacaagaccaaaccatccttcttttttcaaaaccatggattttttaaccaaacagatgtacacagcatcgatgtggtggagcaacaacacagccatattttccttcttcacattttgcaacaatggagatgaatgcaaggcaaatcaaccactatcagatgctgagttcaagttctcatcaaaatctcttggcaaattctctcaatcatccatcatcacagcaactcatgagccaatccgggaaagcaccattcagcttgcgaattaggcctaaacttttccattctcctaatagcagtgaaagccattcccaaaaagatcaggatttcccttccaagtgatgcacaaatagagcaaaataaagttgtcaaaattcttcttctcctcacaaattatgcatggattgatatcttgagggctgtgtgggaattagttgcaaga

SEQ ID NO: 23 - protein, 247 aa, Glycine max
MAKASASSVAQWPRLKDPRIVRVSRAFGGKDRHSKVFTIRGLRDRRVRLSVPTAIQLYDLQDRLGLSQPSKVVDWLLDAAKHEIDELPPLPVVPSVNNFTLGHPSAVTSNEATTSNSQPNEQLLNINRSIQWEEVKRISKEAIMKVLAHLGNNGFANQTDMHSINVVPFPTSTLSLSTGNSQILLCPPGTAQPYFPSHVTMDMDPRQINHYQMLSSAAKAPPTKTKISLPTDTQNTKYIFHGYHGFE

SEQ ID NO: 24 - DNA, 744 bp, Glycine max
atggcaaaggcttcagcgagctcagtagcacaatggccaagattgaaagatccaaggattgtgcgagtatctagagcctttggaggaaaagacaggcacagcaaggttttcacaataagagggttaagagacagacgggtgaggctatcagtaccaactgccatccaactctatgatcttcaagataggctagggctcagtcaacctagcaaggttgttgattggttgctcgatgcagctaagcatgaaattgatgaacttccaccacttcctgttgttccttcagtgaataacttcacccttggtcatccatctgctgttacctctaatgaagctaccacctcaaattctcagcctaatgagcaacttcttaatatcaacagaagcattcagtgggaagaagtgaagaggataagcaaggaagcaataatgaaggtcctagcacacttgggaaacaatggatttgcaaaccaaacagatatgcacagcattaacgttgtacctttcccaacatcaacattgtcattatcaactgggaattctcaaattctgctatgtcctccaggaacagcacaaccatatttcccttcacatgttacaatggacatggatccgagacaaatcaaccactatcagatgttgagttcagcagcaaaagcccctcccacaaagaccaagatttcccttccaactgatacacaaaatacaaagtatatattccacggttaccacgggtttgaataa

SEQ ID NO: 25 - protein, 299 aa, Glycine max
SWSAFRNPRIVRVSRSLGGKDRHSKVCTIRGLRDRRIRLSVPTAIELYDLQEKLGLNQPSKVVDWLLETTKSDIDKLPPLQFPHCFAHHHQQTLLPGTSQFSLGGFYDAAANGGNENHLMATKSTRYWDIDSHSLNNGKDLVESVSNSLKGKSWIKTNEQENHQDGGIGGSSSTHNNREEYSSAHYKLFPIGTTTNNSYLPGLLNNGMTHNSSNHHSEPSSLSLSHFGSHGLFPSSHNAHPSSCSGVPFSSCNFSAASSGPQLLFCPPTSATPASAFYSVESDPRQSNNNVQIFSSSSQ

SEQ ID NO: 26 - DNA, 898 bp, Glycine max
atcatggtcagcattcagaaatccaagaattgtgagagtgtcacgttccttgggaggaaaagataggcacagcaaggtttgcaccattaggggactgagggacagaaggattagactttctgttcccacagcaattgagttatatgatcttcaagaaaagcttggactcaaccaacctagcaaggtggtagattggttgcttgaaaccaccaaatctgatattgataagcttccaccacttcaatttcctcactgttttgctcatcatcaccaacaaaccctactgccaggtacctctcagttttctcttggaggcttctatgatgctgctgctaatgggggaaatgaaaaccacctcatggcaacaaagtcaactaggtattgggatatagattcacattcactaaataatggtaaggatctagttgaaagtgtgtcaaactccctaaaaggaaagagctggatcaaaacaaatgaacaagaaaatcatcaagatggaggaattggtggtagtagtagcactcataacaatagagaggaatattcttcagctcattataagctcttccctattgggacaactactaataactcttatttacctggtttgttaaacaatggcatgacacataactcatccaaccaccattctgagccttcaagtctatctttatcccactttggaagccatggattgtttccttcatcacataatgcccatccaagcagttgcagtggtgtgccattttcatcttgtaatttttctgcagcatcatctggtccccaattgcttttctgtccacctacttctgctacaccagcatctgctttttactcagtggaaagtgatccaagacaatccaacaacaatgttcagatctttagctcaagttcccaa

SEQ ID NO: 27 - protein, 358 aa, Glycine max
EGYQAKQEGDTTTNIDKFSKASSTTSRQWSAFRNPRIVRVSRSFGGKDRHSKVCTIRGLRDRRIRLSVPTAIQLYDLQDKLGLSQPSKVIDWLLEATKFDIDKLPPLQFPQGFGQFHHHPQTLFPFHESSAASHQLSLGPFSDASSTFVRDGGIQNLMAKSRYWENINSMSRLRGNKEAEKGKWIKTSEEENHDQDGVVGGYNNLQVSTQRLFPMGSSTTTTTHSLLPGLLNNSMPYNAYSSEPSSLSLSQFGSHGLFPSQQVDPNPSSGNNGVQFQSSLSLPSSGSQLLFGSSSATPSMFTTYAPFIAPSVDPDPRQFNHIHQFLNSGSQILPPHPLIPSLHHPFNSQIRPFPAALF

SEQ ID NO: 28 - DNA, 1075 bp, Glycine max
gaaggttatcaagcaaagcaagagggtgacacaaccactaatattgacaagttctccaaggcatcttctactacttcaagacaatggtcagcgttcagaaatccaagaattgtgagagtgtctcgttcttttggagggaaagataggcatagcaaggtttgcaccataagagggttgagggacagaaggattaggctctcggttcccacagctattcagctatatgatcttcaagacaagcttggactaagccaaccaagcaaagtgatagattggttgcttgaagccactaaatttgacattgacaagctccctccactccaattccctcagggttttggtcaatttcatcatcatccacaaaccctattcccttttcatgaatcaagtgctgcctctcatcagctgtctcttggacccttcagtgatgctagctccacttttgtgagggatggagggattcaaaacctcatggcaaagtcaaggtattgggagaacataaactcaatgtcaagattaagaggcaataaggaagctgaaaagggcaagtggatcaaaacaagtgaagaagagaatcatgatcaagatggtgttgttggaggttacaacaacttgcaagtttctactcaaaggcttttcccaatgggtagtagtactactactactactcactcccttttacctggtttgttaaacaattccatgccatataacgcctacagttctgagccttctagtttatctttatcccaatttggaagccatggattgttcccttctcaacaagtagatcctaatccaagcagtggcaacaatggtgtgcaattccaatcttctttgtcattaccttcatcgggctctcaattattgtttggctcatcttctgctacaccatcaatgttcaccacctatgctccatttattgcaccctcagtggaccctgatccaagacagttcaaccacattcatcagttcttgaactcaggctctcaaatcttgcctcctcatcctctcataccatctcttcatcacccattcaattcacagatcagacccttcccagcagcactcttta

SEQ ID NO: 29 - protein, 342 aa, Glycine max
QWSAFRNPRIVRVSRSFGGKDRHSKVCTIRGLRDRRIRLSVPTAIQLYDLQDKLGLSQPSKVIDWLLEATKFDIDKLPPLQFPQGFGQFQHPQTLLPFHDSSAASHQLSLGPFCDASSTFVRDGGIQNLMAKSRYWENIDSMSRLRGNKEAEKGKWIKTSEQENHDQDGVVGGYNSLQVSTQRLFPMRSSSTTTTTTHSLLPGLFNNSMPYNSYNSEPSSLSLSQFGSHGLFPTQQVDPNPSSGNNGVQFQSSFPLPSSGSQLFFGSSSATPSMFTTYAPFIAPSVDTDPRQFNHIQFLNSGSHILPHPPLIPSLHHSFNSHVRPFPEAPFSSKLLDSNSSN

SEQ ID NO: 30 - DNA, 1028 bp, Glycine max
acaatggtcagctttcagaaatccaagaattgtgagagtgtcacgttcttttggagggaaagataggcatagcaaggtttgcaccataagagggttgagggacagaaggattaggctctcagttcccacagcaattcagctatatgatcttcaagacaaacttggactaagccaaccaagcaaagtgatagattggttgcttgaagccaccaaatttgacattgacaagctccctccactccaattccctcagggttttggtcaatttcagcatccacaaaccctactccccttccatgattcaagtgctgcctctcatcagctctctctaggacccttctgtgatgctagctccacatttgtgagggatggggggattcaaaacctcatggcaaagtcaaggtattgggagaacatagattcaatgtcaagattaagaggcaataaggaagctgaaaagggcaagtggatcaaaacaagtgagcaagagaatcatgatcaagatggtgttgttggaggttacaacagcttgcaagtttctactcagaggcttttcccaatgcgtagtagtagtactactactactactactcactcccttttacctggtttgtttaacaattccatgccatataactcctacaactctgagccttctagtttgtctttatctcaatttggaagccatggattgttccctactcaacaagtagatcctaatccaagcagcggcaacaatggtgtgcaattccaatcttcttttccattaccatcatctggctctcaattattttttgggtcatcttctgctacaccatcaatgttcaccacctatgctccatttattgcaccctcagtggacactgatccaagacagttcaaccacattcagttcttgaactcaggttctcatatcttgcctcaccctcctctcattccatctcttcatcactcattcaattcacatgtcagacccttcccagaagcaccctttagttcaaagcttttggattcaaacagcagcaacc

SEQ ID NO: 31 - protein, 336 aa, Glycine max
MFLIGSVRRAKSMMIEKGDDDNNIKIEKLLKARPSTSSSSRSWSAFRNPRIVRVSRSLGSKDRHSKVCTIRGLRDRRIRLSVPTAIQLYNLQDKLGFNQPSKVVDWLLEATKSDIDKLPPLQFPHCFAHQFHQQTLLPGTSQFSLGGFYDAANNNSTFIKDGGNQNRLTATKSTRYWDIDSLNNGKELAESVSISQKGQFWIKTNEQENHQGGGIGGSSTTTHNREDNSSVHYKLFPIGTTTNNSYLPGFGIGVPFSSSNFSGASSGPQLLFCPTSATPSAFYSVESDPRQSNNNVQIFSSSSQVMKRLPLIQSLHSTNSPINTLLHINHCENDVL

SEQ ID NO: 32 - DNA, 1011 bp, Glycine max
atgtttctgatcgggtcggtgaggagagcgaaaagcatgatgattgaaaagggtgatgatgataacaacattaaaattgagaagctcttgaaggcacgaccctccacctcctcatcatcaagatcatggtcagcattcagaaatccaagaattgtgagagtctcacgttccttgggaagcaaagataggcacagcaaggtttgcaccattaggggactgagggacagaaggattagactttctgttcccacagcaattcagttatacaatcttcaagacaagcttggattcaaccaacctagcaaggtggtagattggttgcttgaagccaccaaatctgatattgacaaactcccaccacttcaatttcctcactgttttgctcatcagtttcaccagcaaaccctactgccaggtacctctcagttttctcttggaggcttctatgatgctgctaataacaactccacttttattaaggatgggggaaatcaaaaccgcctcacggcaacaaagtcaacaaggtattgggatatagattcactaaataatggtaaggagttagctgaaagtgtgtcaatctcccaaaaaggacagttctggatcaaaacaaatgaacaagaaaaccatcaaggtggaggaattggtggtagtagtactactactcataatagagaggataattcttcagttcattataagctcttcccaattgggacaactactaataactcttatttacctggttttggcattggtgtgccattttcgtcttctaatttctctggagcatcatctggtccccaattgcttttttgtccaacttctgctacaccatctgctttttactcggtggaaagtgatccaagacaatccaacaacaatgttcagatctttagctcaagttcccaagtcatgaagcgtcttcctctcatccaatctcttcattcaaccaattcacctattaataccttgttgcatattaaccattgtgagaacgatgtattataa

SEQ ID NO: 33 - protein, 406 aa, Glycine max
MEEDEIQACNWHYSSRIIRVSRASGGKDRHSKVMTSKGLRDRRVRLSVTTAIQFYDLQDRLGYDQPSKAVEWLIKAASEAISELPSLNNPFPDTPKQPNGDTNYQQNQSQNLSLSKSACSSTSETSKGSGLSLSRSEIRVNRVKARERARERAAKEKEKEKEKESDFNIAHHHHHHNVNNMSHTASFTELLTGGIGSTGSQQQQQQQHHHWSQTVTPMDYFSAGLLVGPSSSASTQQHQSSGQFQLGHGHAHAHALSISPFSGENHSEQMQHFSFMPDHLNNMPSSAASASQHSEGDNNNYNLNFSISSSGLAGYNRGTLQSNSPSSSFLPLLQRFQPLVDGSSNLPFFIGAAPPSSAPPTMDNNNSNNSNNHHHLQFSPIFDGRLQLCYGDGTRQHSDHKGKGKN

SEQ ID NO: 34 - DNA, 1221 bp, Glycine max
atggaggaggatgagattcaagcatgtaattggcactactcttctagaatcataagggtgtctcgagcatctggtggcaaagacaggcacagcaaagtgatgacctcaaaagggttaagagacagaagggttaggctctctgtgaccacagctattcagttctatgaccttcaagatcgcttgggttacgaccaacctagcaaagctgtggagtggctaatcaaagctgcttcagaagctatctctgagcttccttctctcaacaaccctttccctgatacccctaagcagcctaatggagacacaaattaccaacaaaaccaaagccagaatctttctctctccaagtctgcttgtagcagcacttccgagacaagcaagggctcaggcttgtccctctcaaggtctgagatccgtgtgaaccgtgtgaaggcaagggagagggcaagggagagagcagcaaaagagaaggagaaggagaaagagaaagagagtgattttaacattgcacaccaccatcaccaccacaatgtgaacaacatgtctcacactgcttccttcactgagcttctcactggtgggattgggagcacgggaagtcaacaacagcaacagcaacaacatcaccattggtctcaaacagtgacacctatggactacttcagcgcaggcctcttggtaggaccttcatcatctgcaagtactcaacaacaccaatcttcaggacagttccaactgggacatggacatgcacatgcacatgctctttcaatctcaccatttagtggagaaaaccactcggagcaaatgcagcatttttccttcatgccagaccatctcaacaacatgccatcttctgctgcttctgcttctcaacacagcgagggtgacaataacaactacaacctcaacttcagcatctcttcgtcgggccttgctggttacaatagggggacccttcagtccaattctccgtcttcctcgtttttgcctctccttcagaggtttcaacctttagtcgacggatcctccaatctacctttctttataggagctgctcctccttcttctgctcctccaaccatggacaacaacaacagcaacaacagcaacaaccatcaccaccttcagttctcacccatctttgatggccgcttgcaactttgctatggcgatggaacaaggcagcattctgatcacaaggggaagggaaagaactaa

SEQ ID NO: 35 - protein, 532 aa, Glycine max
MEEDEIQACKFPRVGNGRSSDQYQEEEEEGGDLRLRKGVGVGGGGGGGGSDASTNHFHQSSWHHSSRIIRVSRASGGKDRHSKVMTSKGLRDRRVRLSVTTAIQFYDLQDRLGYDQPSKAVEWLIKAASEAISELPSLNNPFPDTPKQLSDKKRPTSGGGQQQQQIQQGHFDDADGDTSYPQNQSQNLSLSKSACSSTSETSKGSGLSLSRSEIRVNRVKARERARERAAKEKEKEKEKEKEKEKESESSIAHHHHHHHVNNNNHMSHTASFTELLTGGIGSTVTVNPNTTTTTTTTTTSPNGSSVHQIHDGHDHETNLFNKGRQQQQQHHWSQTVTPMDYFSTGLLVGPSSSSARTQQHQHQSSSAHFQLGHAHALPISPFSGENHSEQLQHFSFMPDHLNNIPAHHVVTSSSASHQPNGGDNNYNLNFSISTSTGLAGYNNRGTLQSNSPSSSFLPHHLQRFQPLDGSSNLPFFIGAAAPSSAPPTMDNNNNNNSNNHHHLQFSPVFDGRLQLCYGDGTRHSDHKGKGKN

SEQ ID NO: 36 - DNA, 1599 bp, Glycine max
atggaggaggatgagattcaagcatgtaagttcccaagagttggaaatggtaggagtagtgaccaataccaagaggaggaagaagagggtggtgacctaagacttagaaaaggtgttggtgttggtggtggtggtggaggtggaggaagtgatgctagcacaaaccacttccaccaaagtagttggcaccattcttctagaatcataagggtgtctcgagcatctggtggcaaagacaggcacagcaaagtgatgacctcaaaagggttaagagacagaagggttaggctctctgtgaccacagctattcagttctatgaccttcaagatcgcttgggttacgaccaacctagcaaagctgttgagtggttaatcaaagctgcttcagaagctatctctgagcttccttctctcaacaaccctttccctgatacccctaagcaactaagtgataagaaaaggcctactagtggtggtggtcaacaacaacaacaaatacaacaaggccactttgatgatgctgatggagacacaagttacccccaaaaccaaagccagaacctttctctctccaagtctgcttgcagcagcacttccgagacaagcaagggctcaggcttgtccctctcaaggtctgagattcgtgtgaaccgtgttaaggcaagggagagggctagggagagagctgctaaagaaaaagagaaggagaaggagaaggagaaggagaaggagaaagagagtgaatctagcattgcacatcaccaccatcatcaccatgtgaacaacaacaaccacatgtctcacacagcttccttcactgagcttctcactggtgggattgggagcactgtaacagttaaccccaacaccaccaccaccaccaccacaacaacaacaagtcctaatgggtcatcagttcatcagatccatgatggtcatgatcatgagactaatctattcaacaagggaaggcaacaacaacaacagcatcattggtctcaaacagtgacacctatggactacttcagcacaggcctcttggtaggaccttcttcatcatctgcaagaactcaacaacaccaacaccaatcttcttcagcacatttccaactgggacatgcacatgcccttccaatctctccattcagtggagaaaaccactcagagcagctgcagcatttttccttcatgccagaccacctcaacaacataccagctcatcatgtggttacatcttcttctgcttctcatcaacccaacgggggtgacaacaactacaacctcaacttcagtatctctacatcgacgggccttgctggttacaacaatagggggacccttcagtccaattctccgtcttcctcgtttttgcctcatcacctccagaggtttcaacctttagacggatcgtccaacctacctttctttataggagctgctgctccttcttctgctcctccaaccatggacaacaacaacaataataacagcaacaaccatcaccaccttcagttctcacctgtctttgatggccgcttgcaactctgctatggcgatggaacaaggcattctgaccacaaggggaagggaaagaactaa

SEQ ID NO: 37 - protein, 225 aa, Glycine max
MEEEDEIQAQKVRRRRGSDGESTNRFHSSWHHHHHHSSRIIRVSRASGGKDRHSKVMTSKGLRDRRVRLSVTTAIQFYDLQDRLGYDQPSKAVEWLIKSASDSISELPSLNNFPDTPKSDDHEKRANTDVAAGASQNMSDIRVKARERARERTTKEKEKEKNDKLQQHHLVDSNGVHHHQQQQQHLWTTFWGLPLLEQQQHHHTITITIIINILLLVGSHWHKSN

SEQ ID NO: 38 - DNA, 678 bp, Glycine max
atggaggaggaggatgagattcaagcacagaaggtgagaagaagaagaggcagtgatggagagagcacgaacaggttccacagcagctggcaccaccaccaccaccactcttcgaggatcataagggtgtctcgtgcatctggtggcaaagacaggcacagcaaagtgatgacttcaaaagggttgagagatagaagggttaggctttccgtgaccactgccattcagttctatgaccttcaagatcgcttgggttatgaccaacctagtaaagctgtggaatggcttatcaaatctgcttctgattccatttctgaacttccttctttgaacaatttccctgacacccccaaaagtgatgatcatgagaagagagcaaacactgatgttgctgctggggcaagtcagaacatgtctgatattcgtgtgaaggctagagagagggcaagggagagaacaacaaaggaaaaggagaaagagaaaaatgagaagctgcagcagcatcatctggtagacagcaatggtgttcatcatcatcagcagcagcagcagcacctatggactactttctggggccttcctcttctagaacaacaacaacaccaccacacaatcaccatcaccatcatcatcaatattcttcttctggtgggttctcattggcacaaatccaactag

SEQ ID NO: 39 - protein, 465 aa, Glycine max
MEEEDEILQARACKLPRRRGSDGESTNSRFHSSSWHHHNSSRIIRVSRASGGKDRHSKVMTSKGLRDRRVRLSVNTAIQFYDLQDRLGYDQPSKAVEWLIKSASDAISELPSLNNFPDTPQSDDHEKRENTADVAVGVVDGENGNGQNLSLAKSACSSNSETSKGSGLSLSRSDIRVKARERARERTTKEKEKEKEKNDESHRHHHNVVVVPHHNHNVNLVSQTASFTELLTGSDPNKGCDEANNNMMFNKAAGRLQWCSSTTPMDYFLGLSSSRTTTHNHNHLHHHQYSSSSSSGFSLAQIQLGHSLPEAMNHVSAFNNNVSGDNHNHSSSDPHLQQQHLSLIPDHLMSAVVTSSAASSHHHHHHQPSGNDYNLNSTMSSGLAGYNRGTLQSNSPSLLPHSQRFSPIDGSTVPFFIGAASSAAAVAAPAMENNNNNNHHQHQFSSVFDGSRLQLYYGHSDQKGK

SEQ ID NO: 40 - DNA, 1395 bp, Glycine max
atggaggaggaggatgagattcttcaagcacgggcgtgtaagttgccaagaagaagaggcagtgatggggagagcacgaacagcaggttccacagcagcagctggcaccaccacaactcttcgaggatcataagggtgtctcgagcatctggtggcaaagacaggcacagcaaggttatgacttcaaaagggttgagagatagaagggttaggctttctgtgaacactgccattcagttctatgaccttcaagatcgcttgggttatgaccaacctagtaaagctgtggagtggcttatcaaatctgcttctgatgccatttctgaacttccttctttgaacaatttccctgacactccccaaagtgatgatcatgagaagagggaaaacaccgctgatgttgctgttggggtagtagatggtgagaatggcaatggccagaacttgagtttggctaaatctgcttgtagtagcaattctgagactagcaaaggttctggcttgtccctctcaaggtctgatattcgtgtgaaggctagagagagggcaagggagagaacaacaaaggaaaaggagaaagagaaagagaaaaatgatgagtctcatcgtcatcatcacaatgttgttgttgttcctcatcacaaccacaatgtgaacctcgtttcacaaacagcttctttcacggagctactcactggcagtgatcccaacaaagggtgtgatgaggctaataacaacatgatgttcaacaaagcagcaggtagactgcaatggtgttcatcaacaacacctatggactactttctggggctttcctcttctagaacaacgacacacaatcacaatcaccttcatcatcatcaatattcttcttcttcttctagtgggttctcattggcacaaatccaactaggacattctcttcctgaggcaatgaatcatgtttcagcattcaacaacaatgtgagtggtgataaccacaaccactcatcatcagatcctcatctgcagcagcaacacttgtcattgataccggatcatcttatgtcagctgtggttacttcttctgctgcttcttctcatcatcatcatcatcatcagcccagcgggaacgattacaatctgaactccaccatgtcttcgggcctcgctggttacaatagggggacccttcagtccaattctccgtctctgctgcctcactcgcagaggttttcgcctatagacggatccactgtacctttcttcattggagctgcttcttctgctgctgcagttgctgctcctgccatggagaacaacaacaacaacaaccatcaccagcatcagttctcttctgtttttgatggcagccgcttgcaactttactatggccattctgaccagaaggggaaa

SEQ ID NO: 41 - protein, 384 aa, Medicago truncatula
MASSREGNYQAKQEGGDTSMENIEKISKAPSSTSRQWSTSSLRNPRIVRVSRTFGGKDRHSKVCTIRGLRDRRIRLSVPTAIQLYDLQDKLGLGQPSKVIDWLLEATKLDIDKLPPLQFPQSFSQFHPLHSQTLLPFHDQSSNIASHELSLGPLYDPNSTFVGIQNLMARSKFWDIDSRTKGKEVEREYFSEKDKWIKTNEEENQVGEASYNNFHQVSTQKLFPMETHSNFLNNAMSYNNYHSEASSLSLSQFGSSNGLFQSHHQQVDPNQSNGNGLHFPFPFANSQLLFGPSSSSTTPSSSHLSSTPFMNNPVENDPRIQFNHFQFINSSSSQTMPHPLIPSFHHSFNSPPVRPFPIPFSSKLLDSNNNNNSNQLDDNSAPRS

SEQ ID NO: 42 - DNA, 1155 bp, Medicago truncatula
atggcaagttcaagagaaggaaattatcaagcaaagcaagaaggaggtgatactagtatggaaaatattgaaaaaatttcaaaggcaccttcaagtacatcaagacaatggtcaacatcaagtttaagaaatccaagaattgtgagagtatcaagaacatttggaggcaaagataggcatagcaaagtttgtacaataagagggttaagagatagaaggataaggctttcagttccaacagcaattcaattatatgatcttcaagataaacttggtttaggacaaccaagtaaagtaattgattggttacttgaagcaacaaaattagacattgataaactccctccacttcaatttcctcaaagcttttcacaatttcatcctcttcattcacaaaccctacttccttttcatgatcaatcaagtaatattgcttctcatgaactttctcttggtcctttatatgatcctaattccacatttgttggaattcaaaatcttatggcaaggtcaaaattttgggacatagattcaaggactaaaggtaaagaagttgaaagagagtactttagtgaaaaggataagtggatcaaaacaaatgaggaagaaaatcaagttggagaagctagttacaataattttcatcaagtttcaactcaaaagcttttccctatggagactcattctaattttcttaacaatgctatgtcatataacaactaccattcagaagcttctagtttgtctttatctcaatttggaagtagtaatggattgttccaatctcatcatcaacaagtagatccaaatcaaagtaatggaaatggtttacacttcccatttccttttgcaaattcacaattattgtttggtccttcttcttcttctactacaccatcatcatcacatttgagtagtactccttttatgaacaatccagttgaaaatgatccaagaatacaattcaaccattttcagttcataaactcaagctcttcacaaactatgcctcatcctctcattccatcttttcatcactcattcaattcacctccggttagaccttttccgataccatttagctccaagcttcttgattctaataacaataacaatagtaaccaactggatgataatagtgctcctcgttcttga

SEQ ID NO: 43 - protein, 288 aa, Lotus japonicus
ATFYNGKPAIMTETIFKMNKRGPTEAADFPLKQEGLSSHDDDKVAKASAASSSASSQWLKLKDPRIVRVSRAFGGKDRHSKVCTIRGLRDRRVRLSVPTAILLYDLQDRLGLNQPSKVVDWLLNAAKHEIDELPPLPIPPGNITLGYPSLVSPNTTLWKSKPKEVVVTEKDNWVSRNEDEDVKQGSNEDVLPNNVFPIRANHPSFLGLLNTVHPLGYNQFEPSANVGHLANLGFVNQGDIPSNINAVTFPSTLTLSTQPYFPSHVGAMEMDPRQIKHYQMLSSSSQNL

SEQ ID NO: 44 - DNA, 870 bp, Lotus japonicus
tgcaactttctacaatggaaaaccagcaataatgacagaaactatatttaagatgaataaacgcggtccaacagaagctgcagattttccattaaaacaggaaggcctttcaagtcatgatgatgataaggtcgcaaaggcttcagcagcgagctcatcagcatcctcacaatggctgaagttgaaggatccaaggattgtgagagtgtctcgcgccttcggagggaaagacaggcacagcaaggtgtgcacaataagaggactgagagacaggagagtgagactatcagtccccactgccattctcctctatgatctccaagataggcttggactcaaccaaccaagcaaagttgttgattggttgctcaatgcagctaagcatgaaattgatgaactcccacctttgcctattcccccaggaaacatcacccttggttatccatctcttgttagcccaaatacaactctctggaaatcaaagcctaaagaagtagtggttactgaaaaagacaattgggtaagtagaaatgaagatgaggatgttaaacaaggaagcaatgaagatgttttgccaaacaatgtatttccaattagagcaaatcatccatcatttcttggcttgctgaatactgttcatccacttggttataatcagtttgagccttcagctaatgttggtcatttggcaaaccttggatttgtaaaccaaggagatatacccagcaatattaatgctgtgactttcccttcaacattgactttatcaactcagccatattttccttcacatgtgggagcaatggagatggatccaagacaaatcaaacactatcaaatgttgagttcaagttctcagaatttaaccat

SEQ ID NO: 45 - protein, 396 aa, Lotus japonicus
FGKKLYCVSSSVLSSLSLSSLELAQTQKTFFLPHMTENSSKGYDQMKQEGTNEVKTERLISSNKALSTSRQWTAFRNPRIVRVSRALGGKDRHSKVCTVRGLRDRRIRLSVPTAIELYDLQDRLGLSQPSKVVDWLLEASKTDIDKLPPLQIPNCSFSQFHHQDQYSGNDRLSLGVGGLFYDGTSASSQSLMAKSRYNWDVDLVSRLKGKEDESEKGKCLMKETQDSAQKLFPIGINYNSSSSLPGFLNNAMTYNINSFQSEPSSSLSLSQFGSHGVLFPYLQPDSNSMLQYPSNNNGSVVPFSSSLSASQLLFCPSSSATTPTSLFGNPHAPLMTNSSAESSDPRQYNSNHVHTLSSGSSLMPHPFMPSLHPFSNSPLSRRLPTPFSSKLLDSDN

SEQ ID NO: 46 - DNA, 1195 bp, Lotus japonicus
atttgggaaaaaattgtattgtgtttcttcttctgttctctcttccctatctctttcaagcttggaatagttggcacaaacccagaaaactttttttctccctcatatgactgaaaattcaagcaaaggctatgatcaaatgaagcaagagggtactaatgaggttaaaactgagagattgatctcctccaataaggcactctctacctcaagacaatggacagcattcaggaacccaagaattgtgagagtgtcacgggccttgggaggaaaagacaggcacagcaaggtttgcaccgttagaggactccgcgaccgtcggatcaggctttctgtgcccacagcaattgagttatatgatcttcaggacaggcttgggctcagccaacccagcaaggttgtagattggttgcttgaagcctccaaaactgacattgataagcttccaccacttcaaatccctaactgcagcttttctcaatttcatcatcaagatcaatattcaggtaatgataggctttctcttggagttggaggcttgttctatgatggtactagtgcttccagtcaaagcctcatggcaaagtcaaggtataattgggatgttgatttagtgtcaaggctaaagggtaaggaagatgaaagtgagaaaggtaagtgcttgatgaaagaaactcaagactcagctcagaagctctttcctattgggattaattataactcttcttcttctttacctggttttctaaacaatgccatgacatataatattaactccttccaatcagagccttcttcaagtctatctttatcacaatttggaagccatggagtgttgtttccttacctgcagccagattctaattccatgttgcaatatccaagcaacaacaatggcagtgttgtgcccttctcatcctctttgtctgcttcacagttacttttctgtccttcttcttctgctactacaccaacatcactttttggcaatccgcatgctccacttatgacaaactcatcagcggaaagtagtgatccaagacaatacaattccaaccatgtgcacaccttgagctcaggttcctcactcatgcctcatcctttcatgccatctcttcatccattcagcaattcacctttgagtcgacgccttccaacgccatttagttccaagcttcttgattcagataataac

SEQ ID NO: 47 - protein, 307 aa, Oryza sativa
MILGSNQAAAAAAAAAAAEEEAAELARKHTAAVATSRQWSAQTESRIVRVSRVFGGKDRHSKVKTVKGLRDRRVRLSVPTAIQLYDLQDRLGLNQPSKVVDWLLNAARHEIDKLPPLQFPPQDHLCMGHHHHLPSAMPLMHHHGHHHHADDDKYHVAAAAAALAAEKEAAAAGGGGGGGGDDVDGGGGGGAAHIVGRFPAGGYHRFMGLNNPLGMVNSAAGAAMPFHYAGESWNNGSVQDSGAGSPQVAAAAAHHTSPFPSLLSLAPGPHHQLVFYSSEAEQFTVDNLGSQGLSLSSARAFHDQTGS

SEQ ID NO: 48 - DNA, 924 bp, Oryza sativa
atgatactaggaagcaaccaagcggccgccgccgcggctgcggcagctgcggcggaggaggaggcggcggagctagcgaggaagcacacggcggcggtggcgacgtcgcggcaatggtcggcgcagacggagtcgcggatcgtgcgcgtgtcgcgggtgttcggcggcaaggaccggcacagcaaggtgaagacggtgaaggggctgcgcgaccggcgggtgcggctgtcggtgccgacggcgatccagctgtacgacctgcaggaccggctggggctcaaccagcccagcaaggtggtggactggctgctcaacgcggcgcgccacgagatcgacaagctgccgccgctgcagttcccgccgcaggaccacctctgcatgggacaccaccaccacctgccgtcggccatgccgctgatgcaccaccacggccaccaccaccacgccgacgacgataagtaccacgtcgcggcggcggcggccgcgctggccgcggagaaggaggcggcggcggccggcggcggaggaggaggagggggagacgacgtcgacggcgggggcggcggcggagcggcgcacatagtggggaggttccccgccggtggctaccaccggttcatggggctgaacaacccgctggggatggtcaacagcgccgccggcgccgccatgccgttccactacgccggtgagtcatggaataatggcagcgtgcaagacagcggcgccggctcgccgcaggtggccgccgccgcggctcaccacacctcgccgttcccttcactgctttccttggctccagggccgcatcatcagctggtgttctactcctccgaggccgagcagttcacagtagacaaccttggctcgcagggcttgtccctgagctcggcgagagctttccatgatcagacaggaagctaa

SEQ ID NO: 49 - protein, 307 aa, Oryza sativa; identical to SEQ ID NO: 47

SEQ ID NO: 50 - DNA, 924 bp, Oryza sativa; identical to SEQ ID NO: 48

SEQ ID NO: 51 - protein, 307 aa, Oryza sativa; identical to SEQ ID NO: 47

SEQ ID NO: 52 - DNA, 924 bp, Oryza sativa; identical to SEQ ID NO: 48

SEQ ID NO: 53 - protein, 294 aa, Oryza sativa
MTSNNSTNEELGGGGRKAADKPSGGGGAAAAVASSRHWSASTESRIVRVSRVFGGKDRHSKVRTVKGLRDRRVRLSVPTAIQLYDLQDRLGLSQPSKVVDWLINAAQAEIDKLPPLQFPPHDHDLVAAAASSMAPPPFANGGDGHHGASASSMLEDGDKAAGGGGMKAFMSLSNSLGLLNAATMPATLAAHHHHHHHAAAYYAAAESWGNGGNGGHHHDVSHGVSPSAHNSPFPSLLSLAPGSHHQFVFYSPEGGGFAVKEAAAEQFPVDSLDHSQGQLTLSSARSFLHSGSQG

SEQ ID NO: 54 - DNA, 885 bp, Oryza sativa
atgacaagcaataacagcacgaatgaggagctcggcggcggcggcaggaaggcggccgacaagccgagcggcggcggcggcgccgccgccgccgtggcgagctcgcggcactggtcggcgtcgacggagtcgcggatcgtgcgcgtgtcgagggtgttcggcggcaaggaccgtcacagcaaggtgaggacggtgaaggggctccgcgaccggcgggtgcggctgtcggtgccgacggcgatccagctctacgacctgcaggaccggctggggctcagccagccgagcaaggtggtcgactggctgatcaacgccgcccaggccgagatcgacaagcttccgccgctccagttcccgccgcacgaccacgacctcgtcgccgccgccgcctcgtccatggcgccgccgccgttcgcgaacggaggcgacggccaccacggcgcctccgcgtcgtcgatgctggaggacggcgacaaggccgccggcggcggcggcatgaaggcgttcatgagcctgagcaactccctcggcctgctcaacgccgccaccatgccggcgacgttggccgctcaccaccaccaccatcatcacgccgcggcgtactacgccgccgccgaatcttggggcaacggcggcaatggtggccaccaccatgatgtcagccatggcgtctcgccttcggctcacaactcgccgttcccgtcgctgctgtccttggcaccgggctctcatcatcagttcgtcttctactcgccggaaggcggcggattcgccgtgaaagaggctgcagccgagcagtttcctgtagatagcctcgaccactcgcaagggcagctcacactgagctcagcaagaagcttccttcactctggaagccagggatga

SEQ ID NO: 55 - protein, 445 aa, Oryza sativa
MELAGSNNKRGRVRGPNDDDDDAGEPDAKRHHHQLLLPWPQQQQQQHPASRIYRVSRASGGKDRHSKVYTAKGIRDRRVRLSVSTAIQFYDLQDRLGYDQPSKAIEWLIKAAAAAIDKLPSLDTASFPTNPASSAAVAAAAAPPLPHAEREQQQQLTKSGCSSTSETSKGSVLSLSRSESRVKARERARERSSAAAAAASKDAGDDAATPTAPTAAPASSQAASFTELLTGMAAANASPADHKQQQAWQPMTVAAATADYIGFAAAAAPHTQPRKSAAGHHSAMPHTFASPAPHLANITPIAMAPAQHFTLTPAAAEHHAEMTHYSFDHFMPVHAAAAAAAAASTPAGGDYNLNFSMSSGLVGVHSRGTLQSNSQSHLSSHHHHHHQQQQQQQQLQRLSAPLDAPNIPFLFSPAAAPTAADTQFAAALQLWDGFRHADIKEKGKH

SEQ ID NO: 56 - DNA, 1338 bp, Oryza sativa
atggagctcgccggcagcaacaacaagcgggggagggtgagggggcccaacgacgacgacgacgacgccggggagccggacgccaagcgccatcaccaccagctgctgttgccgtggccgcagcagcagcagcagcagcatccggcgtcgcggatctaccgggtgtcgcgggcctccggcgggaaggaccgccacagcaaggtgtacacggccaagggcatccgcgaccgccgcgtccgcctctccgtctccaccgccatccagttctacgacctccaggatcgtctcgggtatgaccagccgagcaaggccatcgagtggctcattaaggccgccgccgccgccatcgacaagctcccttcgcttgataccgcttccttccccaccaaccccgcctcctccgccgctgttgctgctgctgctgctcctcctcttccccatgccgaacgcgagcagcagcagcagctcaccaagtccggatgcagtagcacgtcggagaccagcaagggctccgtcctctccctctcccgctccgagagccgcgtcaaggccagggagcgcgccagggagcggagcagcgcggccgccgccgcggcatccaaggacgccggggacgacgccgccacccccaccgcgcccaccgccgcgcccgcctcctcgcaggcggcttccttcaccgagctactcaccgggatggccgcagccaacgcctcacccgctgaccacaagcagcagcaggcgtggcagccgatgaccgtcgccgcggccaccgccgactacatcggcttcgccgccgccgccgcgccgcatacgcagccgcggaaatccgccgcgggccatcactccgcgatgccgcacaccttcgcatcacccgctccccacctcgccaacatcacccccatcgccatggcgcccgcgcagcacttcaccctcacacccgccgccgccgagcaccacgcggagatgacgcactactcgttcgaccacttcatgcccgtccacgcggcagcagcggcggcggcggcggcctccaccccggccggcggcgactacaacctcaacttctccatgtcctcgggtcttgtgggtgtccacagtagggggacccttcagtccaattcgcagtctcacctctccagccatcaccaccaccatcaccagcaacagcagcagcagcagcagctgcagaggctgtctgctccgctggatgcgcccaacattccgttcctcttcagcccggccgccgcgcccaccgccgcggacacccagttcgccgccgcattgcagctctgggacgggttccggcacgccgacatcaaggagaagggcaagcactga

SEQ ID NO: 57 - protein, 357 aa, Oryza sativa
MEAAVGDGEGGGGGGGRGKRGRGGGGGEMVEAVWGQTGSTASRIYRVRATGGKDRHSKVYTAKGIRDRRVRLSVATAIQFYDLQDRLGFDQPSKAIEWLINAASPAIDTLPSLDPAAFAAIPHAAADAAPTRRRSQQQQQQLSNKSGCSSTSETSKGSDKEVTVASAPAQAASFTELLIAGVAASSAGGGAIGNGADCVGIAHPGKGGAEGASTYGFSAASSFGDAPPIGMVPAPPFNFSAPGADMAAHYSLAQDQLAAPPPPAGGDYNLNFSMSSGFLGANRGTLQSNSPSNMSGHHHHHHQQQLQRLDGSTISFLLGHAAAAAHPAASEGQITSTAALQLWDGFRHSGMKEKSKN

SEQ ID NO: 58 - DNA, 1074 bp, Oryza sativa
atggaggcggcggtgggggacggggaaggcggtggcggcggcggcgggcgggggaagcgtgggcggggaggaggaggaggggagatggtggaggcggtgtgggggcagacggggagtacggcgtcgcggatctacagggtgagggcgacgggggggaaggacaggcacagcaaggtgtacacggcgaagggaatccgcgaccgccgcgtccgcctctccgtcgccaccgccatccagttctacgacctccaggaccgcctcggcttcgaccagccgagcaaggccatcgagtggctcatcaacgccgcctcccccgccatcgacaccctcccgtccctcgaccccgccgccttcgccgccattccccacgccgccgccgacgccgcgcccacgcggcggcgttcgcagcagcagcagcagcagctcagcaacaagtccggatgcagtagcacgtcggagaccagcaaggggtcggacaaggaggtcaccgtggcgagcgccccggcgcaggccgcgtcgttcactgagctgctcattgctggggtggcggcgtcgtcggcggggggtggcgccattggcaacggcgccgactgcgtgggcatcgcgcaccccgggaaaggaggcgcggagggggcgtccacgtacggcttctccgccgcctcgagcttcggcgacgcgccaccgatcggcatggtgccggcgccgccgttcaacttctccgcccccggcgccgacatggcggcgcactactcgctcgcccaggaccagctggcggcgcctccgcctcctgccggaggcgactacaacctcaacttctccatgtcctcgggtttcttgggtgccaatagggggacccttcagtccaattcgccgtcgaacatgtccggccaccaccaccaccaccatcagcagcagctccagaggctggacggctccaccatttccttcctgcttggccacgccgccgcggcggcgcacccggcggcgtccgagggccagatcaccagcaccgccgcgttgcagctgtgggatggattccgccactccggcatgaaggagaagagcaagaactga

SEQ ID NO: 59 - protein, 588 aa, Physcomitrella patens
MMFRLPQPGGKEEEQRRQRLSEAEPARQGQPQQQQQIAGYEISISGTEHSEKVEGLSKRGGGEGSSEERRVRARPSDQPSKSGPPGAECTRPARAARSSGGKDRHSKVNTAKGPRDRRVRLSVPTAVQFYDVQDRLGFDQPSKAVEWLIKHAKAAIDELEQLPSVRQGEPAASFEQQPQTTSSTVSTFSAGLPGVLAAPLPESSSTSYGTYDSHGQPAMMSTFGHTGHGMEAVMGPQHQHHHLASYGLNFPLEQQAGLFRAGSSGDSQAGGSGTARVESRAKARERARERAKEKHITERDPGTGRISSSMVTHHVPASASAGMQQQHPHLVQSPYANLPSQVSSALLHPFQASPAFNASTGIHPPSRPPPPAATPYHASSYGFSESSFVYSGHYNPPPTPYNQAEPSLHQQQQQLPSVSHYFVENPPPPSHYSSSNPQGMNTPKPMTLPGFSPGPASSSLRASLFGSVISEQHQSLHYSSGSYLSPSGYYFPPSYASIAQRPPPSSSPLLGPSLPGSSLRPSQGTMRYTLPGMRSDQEQARGHEQQQQQEEELGTHDQTPHSLDPNFYTSQIPARLQGLEELEEEFKR

SEQ ID NO: 60 - DNA, 1767 bp, Physcomitrella patens
atgatgtttcgactgccacagccaggcgggaaagaggaagaacaacgtcgtcaacgcttatcggaggcggagccagctaggcagggtcagccacagcagcagcagcagatagctggctacgaaatatcaatctctggcaccgagcattcggaaaaagtggaagggctgtccaaacgagggggcggtgagggatcatctgaggagagaagagtgcgtgcgcgaccaagtgaccaacccagcaagagtggtcctccaggggccgagtgtacccgcccggcccgagcggcacgatcctctggagggaaggatcgtcatagcaaagtcaacacagcgaaagggcccagagaccgaagagttcggctgtcagttcccaccgctgttcaattttacgatgtgcaagaccggcttggcttcgaccagcccagcaaagcagtggaatggttgatcaagcatgccaaagccgccattgatgagctcgagcagcttccgtccgtgcggcagggggagcctgcagcctcgtttgagcaacaacctcagaccactagctccaccgtctccaccttctcagctggccttcccggggtcctcgctgcccccttacccgaatcctcaagcacatcatatgggacctacgacagtcatgggcagccagcaatgatgtctactttcggccacactggtcacggaatggaagcagtcatgggtccccaacatcaacaccatcacctcgcaagctatggtctgaattttccactggaacagcaagctggattgttccgggcaggaagctctggcgacagtcaagctgggggctccggcaccgcccgtgtggagtctcgagccaaagcccgcgagcgtgctcgggaacgagccaaggagaaacacatcacggagagggatccaggaactggaagaatatcctcaagcatggttacgcatcatgtgcccgcatccgcatccgcaggaatgcagcagcaacatccccacctcgtccaatccccgtacgcgaacctcccctcacaggtgagcagcgcgcttctacatccatttcaagcgtcccctgccttcaatgcttccactggtatccaccctccctcacggcctccccctcctgctgctactccttaccatgcatccagctacggtttctctgagtcttccttcgtatacagcgggcattacaatcctccccccacaccctacaaccaagctgagccatccttgcatcagcagcagcagcaactgccctctgtctcccattattttgtcgagaatcctccaccgcccagccattacagcagcagcaacccccagggaatgaacacccctaaacccatgacgttgcctgggttctctcccggccctgcctcctcctcccttcgagccagcctattcggaagcgtaattagcgagcagcatcaatctctccactattccagtggcagctacctatcgccgtctggatattatttcccaccctcgtatgcgtcgatcgcccaacgcccgccgccgtcctcatccccgttgttaggcccatctctcccgggcagcagtcttcgtccctcgcaaggaaccatgaggtataccctcccggggatgcggtccgaccaagagcaggctcgaggccacgagcagcagcagcagcaggaggaggagcttgggacccacgatcagacaccccatagcctggatcccaacttctacacctcacagatacctgcaagattgcagggcttggaagagcttgaggaggagttcaagcgttga

SEQ ID NO: 61 - protein, 365 aa, Populus trichocarpa
MITSSKQVDSPTKKEEDSNDGKISKASSSSAPWLRLKDPRIVRVSRAFGGKDRHSKVCTIRGLRDRRVRLSVPTAIQLYDLQDRLGLNQPSKVVDWLLNAAKHEIDELPPLPMPPGNFSLNHQAMLGSSNEVGASRSSKEVFKMNNGVDWEDTSGFTRPSFWNADAILRAKSKEVMTDPVNEKENWAKRDEEDKEDGSIEGNTAQALSSSFLQRASHSSLPGLINNAMPYGSFFHLEPQSFPLSHMGNHGFATQTGDIHNLNVVPLSSALSLSSGSQILVCPPGTTQSYFPSHATASMEIDPRQINHFQWLTPSTQNLLPNSLTPSPYPISQSMRPSQYFSVTPRVLHSQNSESPPDKDPQFPCK

SEQ ID NO: 62 - DNA, 1098 bp, Populus trichocarpa
atgattactagttcaaagcaagtggattctccaacaaaaaaggaagaggattcaaatgatggaaagatttctaaggcttcatctagctcagcaccatggttaaggttgaaggatccaagaattgtgcgagtgtcgcgtgctttcggaggaaaagacaggcatagcaaagtttgcaccattagagggttgagagataggagagttaggctttcagttcccactgctattcagttgtatgatcttcaagatagacttggccttaatcaaccaagcaaggtagttgattggttgcttaatgcagccaagcatgaaattgatgaactccctccactcccaatgccaccagggaacttttccctgaatcatcaagcaatgctgggttcttctaatgaagttggcgcctcacggtctagcaaagaagtgtttaagatgaacaacggcgtcgattgggaggatacaagtggatttactagaccgagtttctggaatgctgatgcaattttgagggctaagtcgaaagaggttatgacagatccagtgaatgagaaagaaaattgggcaaaaagagatgaagaagataaggaggatggcagtattgaagggaatactgcgcaagctttatctagcagtttcttacaaagagctagtcattcttccttaccaggtttgataaataatgccatgccttatggttctttctttcatttagaacctcaaagttttccattatctcatatgggaaaccatggatttgcaacacaaacaggagatatacacaacctaaatgtcgtgccattgtcatctgccttgtctttatcatctggctctcaaattttagtgtgtccacctgggacaacacagtcttatttcccttcacatgccacagcttccatggagattgatccgagacaaatcaaccactttcaatggttgaccccaagcactcaaaatctcttaccaaattcccttaccccaagtccgtaccctataagccaatccatgagaccttcccagtatttcagtgtgactcctagggttctccactctcagaatagcgaaagtccaccggacaaggacccacaatttccttgtaaatga

SEQ ID NO: 63 - protein, 333 aa, Populus trichocarpa
MITSSKEVDCPPKKEDDTNDGKISKASSSSAPWLRLKDPRIVRVSRAFGGKDRHSKVCTIRGLRDRRVRLSVATAIQLYDLQDRLGLNQPSKVVDWLLNAAKHDIDELPPLPAIPGNFSLNHQAMLGSSRELGASQSGNSINWEDPSAFPRPNLWNADAFWRAKSKEVVMDPVNEKENWTKRIEEDKQDSNIEGNSTAQVSSSSFLHRASHSSLPSLINNAMPYGSFFHLEPPNFPLSQMGNHGFSTQTGDIHNLNAVPLSSALSLSSGSQFFVCPPGTTQSYFPSHVTASMENDPRQINHFQVLTPSTQNLFPNSLTPSPYHTVFPMVLSQL

SEQ ID NO: 64 - DNA, 1002 bp, Populus trichocarpa
atgattactagttcgaaagaggtggattgtccaccgaaaaaagaggatgatacaaatgatggtaagatttctaaggcttcatccagctcagcgccatggttaagattgaaggatccaagaattgtgcgggtgtctcgagctttcggaggaaaagacaggcacagcaaagtttgcaccattagagggttgagagataggagagttaggctttcagtagccactgctattcaattgtatgatcttcaagataggcttggacttaatcaacctagtaaggtggttgattggttgcttaatgcagctaagcatgatattgatgaactccctccactcccagcgataccagggaacttttcccttaatcatcaagcaatgctaggttcttctcgtgaactcggcgcctcacagtctggcaacagcatcaattgggaggatccaagtgcatttcctagaccgaatttatggaatgctgatgcattttggagggctaagtcgaaagaggtggtaatggatccagtgaatgaaaaagaaaactggacaaaaagaattgaagaggataagcaggatagcaatattgaagggaatagtactgcacaagtttcatctagcagtttcttacacagagctagtcattcttccttgccaagtttgataaataatgccatgccatatggttctttctttcatttagaacctccaaattttccattatctcaaatgggaaaccatggattttcaacacaaacaggagatatacacaacctaaatgctgtgccgttgtcatccgccttgtctttgtcatctggctctcaattttttgtgtgtccacctggtacaacacagtcttattttccttcacatgtcacagcttccatggagaatgatccgagacaaatcaaccactttcaagtgttgaccccaagcactcaaaatctctttccaaattcccttaccccgagtccataccatacagtttttcctatggtactctcacagctataa

SEQ ID NO: 65 - protein, 377 aa, Populus trichocarpa
MISNSRDMDLQGKQAGGGGSEGDKISKATPSTSSRQWSAFRNPRIVRVSRSFGGKDRHSKVCTVRGLRDRRIRLSVPTAIQLYDLQDRLGLSQPSKVIDWLLDATKNDIDKLPPLQMPPGFGQFHQPMLFSHQSNIASPFFDPNSTFIKDVGFHSLGIKIGTSTAGLDGQVQNSSNTMPKSSYSRDIEASIRAKSKQVDTINEKGKWIKTNEDGEIGSYSTTGQVPAQNFFPLANHSSLPSLLYNPTVPFNSNHWDASNLSLSTQFGGHGFLSQTENSLDTPSSLPLSSGSQLFFCPPPPMPPLFPSYPPCVTTPSENESRDMNHFQLLSSSSSQHILPISLTMSSTTKPFSLNLNPGLLRSQNNNESNPDEDTTES

SEQ ID NO: 66 - DNA, 1134 bp, Populus trichocarpa
atgatttcaaattcaagagatatggatttgcaaggaaaacaagcaggtggcggcggcagtgagggcgacaagatttccaaggcaacaccatccacttcatcaagacaatggtcagcatttagaaatccaaggattgttcgcgtctcccggtcttttggaggaaaagataggcatagcaaggtttgcacagtgaggggactgagggaccggcgaattaggctttctgtacccacagccattcagttatatgatcttcaagataggcttggccttagtcagcctagcaaggtaatagattggttgcttgatgctactaaaaatgatatcgataagttgcctccacttcagatgcctccagggtttggtcaattccaccagccaatgctattttctcatcaatcaaatattgcatctcccttctttgatcccaattctacgtttatcaaggacgtaggatttcattcattagggataaagatcggtaccagtactgctggtcttgatgggcaggtgcaaaacagtagtaatactatgccaaaatcaagctatagcagggatatagaagcttcaataagagcaaaaagcaagcaagttgacactattaatgagaaaggaaagtggatcaaaacaaatgaagatggagaaattggaagttacagtactactggacaggtccccgctcaaaatttctttcctttagccaatcattcttccttaccaagcttgctatacaatcctactgtgcccttcaactccaatcattgggatgcttcaaatttatctctatctacacaatttggaggccatggatttctctcccaaacagaaaattccttggacaccccatcatcactgcctctctcttctggctctcaattatttttctgcccacctccaccaatgccaccactctttccatcctatcctccttgtgttaccactccctcagagaatgagtccagagatatgaaccatttccaattattgagctcaagttcgtcacagcatatcttaccgatttccctgacaatgagctcaacaacaaaacctttttccttgaatctaaatcctgggcttcttcgttcgcaaaataataacgaaagcaatcctgatgaagatactacagaatcataa

SEQ ID NO: 67 - protein, 473 aa, Populus trichocarpa
MEAEEIQRQPCKFSRVGNGRNDSSRIAQNGYGDHQYPDEEEDGGVKRSNPNNGGGAGGTINRLREWHHSSRIIRVSRASGGKDRHSKVWTSQGLRDRRVRLSVTTAIQFYDLQDRLGYDQPSKAVEWLIKAAQDAINELPPLNHSFPDTPKQLSDEKRTSDGTEQGFDSADVELNDPNFSQSQNQNQHLSLSKSACSSTSETSKGSGLSLARSGIRDSRVKARERARERTAKEKENDSRIAHHHNINPISQNSTFTELLTGGIGSVSNNNNSNSSSSNNNNAASPSEANLFQKAAAARQWLSTPMDYISTGLLGPSSSRTTHHSSGFPGQIQLGNSIPQPMTMSVPPFNVSGENHQEQLQHFPFISDHLMPVAATTQTVGDYNLNFTISSSLAAGFNRGTLQSNSSSPSLFSHLQRFSTSSPIDGSTTNVPFLIGAPQQAMENHHHQHQFPHGLQLCYGDGTRHSDQKGKGKN

SEQ ID NO: 68 - DNA, 1422 bp, Populus trichocarpa
atggaggcggaggagattcagagacaaccttgtaagttctctagagttggcaatggaagaaatgactccagcagaatagctcaaaacgggtatggtgaccaccaatatcctgatgaagaagaagatggtggggtcaagaggtctaatcctaacaacggtggtggtgctggtgggaccatcaatcgtcttcgtgagtggcaccattcgtcacgcataattagggtttctagagcatctggtggaaaagatcggcatagcaaggtatggacctcacaggggttaagggaccggagagtacgtttatcggtgaccacagccattcaattctatgatctccaagatcggttgggttatgatcaaccaagtaaagctgtagaatggctgattaaagcagctcaggatgcaatcaatgagctccctccactcaaccattcgtttcctgacactccaaagcagttaagcgatgagaaaagaacaagtgatggtaccgagcaaggatttgattcagctgatgttgagcttaatgacccaaattttagccagagccagaaccagaaccagcatctctctctatctaaatctgcttgcagtagcacttctgagacgagcaaaggttcagggttatcactcgctcgatctggtattcgtgatagtcgtgttaaggctcgtgagagagccagagagagaacagccaaggagaaagagaatgattctcgtattgcacatcaccacaacataaaccccatctctcaaaactccacgtttactgagcttttaactggggggattggtagtgttagcaacaacaacaacagcaacagcagcagcagcaacaacaacaatgctgctagtccttctgaggcaaatttgttccaaaaagcagctgctgcaaggcagtggctttcgacccccatggattacatcagcacagggcttttagggccctcctcgtcacggacaactcatcattcatcggggtttccagggcaaatccagttagggaactcaatcccacaaccaatgacaatgtcggtcccgccattcaatgtatcaggagagaatcaccaagagcagttgcaacattttcccttcatttcagatcatttgatgccagtggcggccacaacacaaacagttggggattataatctgaattttacaatatcttcaagccttgctgctggtttcaacagggggacccttcagtccaattcatcgtcaccttctcttttctctcacctccagaggttttctacttcttctcccatagacggatccaccacaaacgtacctttccttattggtgcaccacaacaagctatggagaatcaccaccaccaacatcagttcccacatgggttgcagctctgttatggcgatggaaccaggcattctgaccagaaaggcaaagggaagaactga

SEQ ID NO: 69 - protein, 478 aa, Populus trichocarpa
MEAEEIRRQPCKFSRVGNGINDASRIAQKGNGTDHQYPDDEEDGELKRTNSNNGAGCGGGGIDAAGGTFNGLRAWHHSSRIIRVSRASGGKDRHSKVWTSKGLRDRRVRLSVSTAIQFYDLQDRLGYDQPSKAVEWLIKAAQDAINELPSLNHSFPDTPKQLSDEKKTSDSTEQGFDSADVELEDPNFRQNQNLSLSKSACSSTSETSKGSGLSLSRSVSRDKARERARERTAKEKEKENDSRIAHHHNINPISQNSTFTELLTGGIGSVRNNSSSNNNNNSTASPTGSDANNLFQKAAAARQWPSSPMDCFGSGLLGPSSSRSTHHSSGFPGQIQLGNSIPQAMTMSIPPFCVSGENHQEHLQHFPFVSDHLIPVAATTQPPASSGDYNLNFTISSGLAAGYHRGTLQSNSSSPSLLPHLQRFSTSSTIDGSTTNVPFFIGAAAPQAMENHHQFPPGLQLCYGDGTRHSDQKGKGKN

SEQ ID NO: 70 - DNA, 1437 bp, Populus trichocarpa
atggaggcggaagagattcggagacagccttgtaagttctctagagttggtaatggcataaatgacgccagcagaatagctcaaaaaggcaatggtactgaccaccaatatcctgacgatgaagaagatggtgagctcaagaggactaattctaacaacggtgctggttgtggtggtggcggtattgatgctgctggtgggaccttcaatggtcttcgtgcgtggcaccattcgtcgcgcataattagggtttcaagagcatctggtggaaaagatcggcacagcaaggtatggacttcaaaagggttaagagaccggagagtccgtttatcagtgagcactgccattcaattctacgacctccaagatcggttgggttatgatcagccaagtaaagctgtagaatggctgatcaaagcagctcaagatgcaattaacgagctcccttcgctcaatcattcttttcctgacacgccaaagcaattaagcgatgagaaaaaaacgagtgatagcactgaacaaggatttgattcagctgatgttgagcttgaagacccaaattttaggcagaaccagaatctctcgctatctaaatctgcttgcagtagcacttcagagacaagcaaaggttcagggctatcactttcgcgttctgttagtcgtgataaggctcgtgagagagccagggagagaacagccaaggagaaggagaaagagaatgattcacgcattgcgcatcaccacaacataaaccccatttctcaaaactccacatttactgagcttttaactggtgggattggtagtgttagaaataacagcagcagcaacaacaacaataacagtactgctagtcctactggttctgatgcaaataatttgttccaaaaagcagccgcggcaaggcagtggccttcgtccccaatggactgcttcggctcaggacttttagggccatcctcgtcgcggtcaactcatcattcatcggggtttccagggcaaatccagttagggaactcaatcccacaagcaatgacaatgtcgatcccaccattttgtgtatcgggggagaatcaccaggagcatttgcaacattttcctttcgtttcagatcatttgatcccagtggccgccacaacacaaccgccggcatccagcggggattataacctaaatttcacaatatcttcaggccttgccgcaggttaccatagggggacccttcagtccaattcatcatcaccgtctcttttgcctcacctccagaggttttctacgtcttctaccatagacggatctaccacaaatgtacctttctttatcggtgctgcagcaccgcaagctatggagaaccaccaccagttcccacctgggttgcagctctgctatggcgatggaaccaggcattctgaccagaaaggcaaagggaagaactga

SEQ ID NO: 71 - protein, 298 aa, Sorghum bicolor
MISGNLTNEELGAGCTKAEGPTAGSGAGAVVAKSPVLSSSRHWPPSTESRIVRVSRVFGGKDRHSKVRTVKGLRDRRVRLSVPTAIQLYDLQDRLGLSQPSKVVDWLLDAAQHEIDKLPPLQFPPQAQDLVAHLPPSMVAPFSNVAAAADRAAAANASAAAMADGDKRHCHGGGIKGLMGLNNSIGLVNATMPLAHGLYYTPAGESWTTNGNNAAVHDHQVSHHGTSPQTTVAHHSPFSSLLSLAAPGPQLVFYSPEGGGFAMKEAADHQFPVDSLEHSQGQLSLSSARSFLHPGNQG

SEQ ID NO: 72 - DNA, 897 bp, Sorghum bicolor
atgataagcggcaatctcaccaatgaagagctgggggccgggtgcaccaaggccgaggggccgaccgcgggctccggcgccggcgccgtcgtcgcaaagagtcccgtgctgtcgagctcccggcactggccgccgtcgacggagtcgaggatcgtgcgcgtgtcgcgggtcttcggcggcaaggaccggcacagcaaggtgaggacggtcaaggggctgcgcgaccggcgcgtgcggctgtccgtgccgacggcgatccagctctacgacctccaggaccggctgggcctcagccagccgagcaaggtggtggactggctgctggacgccgcgcagcacgagatcgacaagctgcctccgctccagttcccgccgcaagcgcaggacctcgtcgcccatctgccgccgtccatggtggcgcccttctccaacgttgcagctgcggccgaccgggccgccgccgcgaacgcgtccgcggcggcgatggccgacggcgacaaacgccactgccacggcggcggcatcaaagggctcatgggcctcaacaattccatcggccttgtcaacgccaccatgccgctggctcacggcttgtactacacgcccgcgggtgagtcttggacgaccaatggcaataatgccgccgtccatgatcatcaggtgagccaccacggcacttcgcctcagacgacggtggctcaccactcgccgttctcatctctgctgtctcttgctgctccggggcctcagctcgtcttctactcgccggaaggcggcggcttcgccatgaaagaagccgccgatcatcagttccctgtagatagcctcgaacattctcaaggccagctctcactgagctcagcaagaagcttccttcaccccggcaaccagggatga

SEQ ID NO: 73 - protein, 289 aa, Sorghum bicolor
MIRGGNNQEAADVEELLAHHQQNKQHAAVATSSRQWSAQTESRIVRVSRVFGGKDRHSKVKTVKGLRDRRVRLSVPTAIQLYDLQDRLGLNQPSKVVDWLLNAARHEIDKLPPLQFPPHQDLMVGHLTPHMPLVHEETKFGHIAAAAALASDGGGAKVSQQGDVDVDVDGTGGAHHLVGRFPGGGYYHRFMELNSALGMVSSAMAPYSNYTGEAWNNSSGGMHDGGAGGSPQVVAAAHHSPFPSLLSLAPRPHQLAFYSSEGDQQFQVENLGSQSLSLSSARAYHDQAS

SEQ ID NO: 74 - DNA, 870 bp, Sorghum bicolor
atgataaggggcggcaacaaccaggaggcggcggacgtcgaggagctgctagcccaccaccagcagaacaagcagcacgccgccgtggcgacgtcgtcgcggcaatggtcggcgcagacggagtcgcgcatcgtgcgcgtctcccgcgtcttcggcggcaaggaccgccacagcaaggtgaagaccgtcaaggggctgcgcgaccgccgggtgcgcctctccgtgcccacggcgatccagctctacgacctgcaggaccgcctcggcctcaaccagcctagcaaggtcgtcgactggctgctcaacgccgcgcgccacgagatcgacaagctgccgccgctgcagttcccgcctcatcaggacctcatggtcggccacctcacgccccacatgccgctcgtgcacgaggagacgaagttcggacacatcgccgcggccgcggcgctggcgagcgacggcggcggcgccaaggtcagccagcagggcgacgtcgacgtcgatgtcgacgggaccggcggagcccaccacttggtggggaggttccccggcggcggctactaccaccggttcatggagctgaacagcgcgttgggcatggtcagtagcgcaatggcgccgtatagcaactacaccggagaggcatggaataacagcagcggcggcatgcatgacggcggcgcaggcggctcgccgcaggtcgtggcagcggctcaccactcgccgttcccgtcgttgctctccttggctccacgtccgcatcagctggcgttctactcctcggaaggtgatcagcagtttcaggtggagaaccttggctcgcaaagcctgtccttgagctcggcgagggcttaccatgatcaggctagctag

SEQ ID NO: 75 - protein, 461 aa, Sorghum bicolor
MEVEAVGCAGAAVAGGGGAGGKRGRAKAGAGAEPEPKRRAVAVAVPPAAGAPWAQQHPASRIYRVSRASGGKDRHSKVYTAKGIRDRRVRLSVATAIQFYDLQDRLGYDQPSKAIEWLIRAAADAIDKLPSLDAAAGFPAHPASAAKDHHAHHGPPPAGDDGDEHHHHQQLTRSGCSSTSETSKGSVLSLSRSESRVKARERARERSAVARDKDKDKDPAAAANDGTARRVVATTTSAQAASFTELLTGMAAASAAAPADHKHHQQSSVSWGQTTTVSAATADYIGFAQPRKGAGHAMAHAFAAPAPHLGNIASIAMAPAAQHFGLASGAVTVASGGGEPNAEMTHFSFLQDHFMPVHAAPAPAPTPAIEYNLNFSMSSGLVGVNSRGTLQSNSQPHISGHHHHQQQLQRLSTPLDAPNIPFLFSPPAAAAAAPTTAEGQFAAFQLWNGFQHADMKEKGKN

SEQ ID NO: 76 - DNA, 1386 bp, Sorghum bicolor
atggaggtggaggccgtgggctgtgcaggcgccgccgtcgccggcggcggcggcgcggggggcaagcggggccgggcgaaagctggcgcgggagcggagccggagcccaagcgacgcgcggtagcggtcgccgtgccgcccgccgccggtgcgccgtgggcgcagcagcacccggcgtcgcggatctaccgcgtctcgcgggcgtcggggggcaaggaccgccacagcaaggtgtacacggccaagggcatcagggaccgacgcgtccgcctctccgtcgccaccgccatccagttctacgacctgcaggaccgcctcgggtacgaccagcctagcaaggccatcgagtggctcatcagggctgccgctgacgctatcgacaagctcccttcgctcgacgccgccgcggggttccccgcgcacccggcctccgccgccaaggaccaccacgctcaccacggcccgccgcctgctggtgacgacggcgacgaacaccaccaccaccagcagctcacgaggtccggatgtagcagcacgtccgagacaagcaagggctcggtcctctcgctctcccgctccgagagccgggtcaaggcaagggagcgcgctagggagcggagcgccgttgccagggacaaggacaaggacaaggaccccgccgcggccgccaacgacggtaccgccaggcgcgtcgtggctaccactacctcggcgcaggcggcgtccttcaccgagctgctcacggggatggccgcggccagcgccgccgcccccgcggaccacaagcaccaccagcagtcgtcggtgtcctgggggcagacgacgactgtctccgctgccaccgccgactacatcgggttcgcgcagccccggaagggcgcggggcacgcgatggcgcacgccttcgccgcccccgctccccacctcggcaacattgcgtccatcgccatggccccggcggcgcagcacttcggcttggccagtggtgccgtcaccgtcgcctctggcggcggcgagccgaacgctgagatgacgcacttctcgttcttgcaggaccatttcatgcccgtccacgcggcgccggcgccggcgccgactcccgccatagaatacaacctcaacttctccatgtcctcgggtcttgtgggtgtcaacagtagggggacccttcagtccaattcgcagccgcacatctccggccaccaccaccaccaacaacagctccagaggctgtctactccactggacgctcccaacatacccttcctgttcagcccgcccgccgctgccgctgcagcgcccaccacggccgagggccagttcgccgcgttccagctctggaacgggttccagcacgcagacatgaaggagaagggcaagaactga

SEQ ID NO: 77 - protein, 389 aa, Sorghum bicolor
MEASAVGDGAAGEGAAPAGRSQQGAKRGRGGAVRGSEADAAWGQPPMLPPPQAPGAGSRIYRVRASGGKDRHSKVYTAKGIRDRRVRLSVPTAIQFYDLQDRLGFDQPSKAIEWLINAASSAIEKLPELDPAAFAALPAPGDADAGGKDKQQQGSKSAGSSTSETSKGSELSLSRSDGRGAGAAARDREVTVASTSAQAASFTELLTGVASAGSITAAEHKQSWHQQPNVSAAAAADCVGIANHGKGAHGLSTHTFSAPAAAKYGNAPPFGLVPAQPFNFTSPIEMPHFSLGQDTLAASSPAAGDYSLNFSMSSGFLGANRGTLQSNSQSNFSGHNHQHLQRLDGPILFGHAHAAAAAHPASENQLTASAALQLWDGLRHSGMKEKSKN

SEQ ID NO: 78 - DNA, 1170 bp, Sorghum bicolor
atggaggcatcagccgtgggcgacggagcagcgggcgagggcgcggcgcccgcgggccgctcccagcagggtgccaagcgcggccgcggcggcgccgtgaggggcagcgaggcggatgcggcgtggggccagccgccgatgctcccgccgccgcaggcgccgggggcgggctcccggatctaccgcgtgcgggccagcggcggcaaggaccggcacagcaaggtgtacacggccaagggcatccgcgaccgccgcgtgcgcctgtcggtgcccaccgccatccagttctacgacctgcaggaccgcctcggcttcgaccagcccagcaaggccatcgaatggctcattaacgccgcctcctccgccatcgagaagctcccagagctcgacccggccgccttcgctgctctccctgcgcccggcgacgcggatgccggcggcaaggacaagcagcagcaggggagcaagtccgcgggcagcagcacgtcggagacgagcaagggctccgagctctcgctctcgcgctccgacggccgcggggccggcgccgccgcgcgggaccgggaggtcaccgtggccagcacttcggcgcaggcggcgtccttcaccgagctgctcaccggggtggcgtcggccggctccattaccgccgccgagcacaagcagtcctggcaccagcagcccaacgtctccgcggccgccgccgctgactgcgtcggcattgcaaaccacgggaaaggtgcgcacgggctgtcgacgcacaccttttctgcccccgccgccgccaagtatggcaatgcgcctccgttcggcctggtgccggcgcagcccttcaacttcaccagccccatcgagatgcctcacttctcgctcggtcaggacaccttggcggcgtcctcgcctgccgccggggactacagcctcaatttttccatgtcctcgggtttcttgggtgccaatagggggacccttcagtccaattcgcagtccaacttctcaggccataatcaccagcacctccagaggctggatggcccgatcttgtttggtcacgctcatgctgcggcggcggcgcatcccgcatccgagaaccagcttacggcatccgcggcgttacagctgtgggacgggctccggcactccggcatgaaggagaagagcaagaactga

SEQ ID NO: 79 - protein, 302 aa, Sorghum bicolor
MEEVAVAAGMERAAGCKRPRGALDGGGSASAAAWRTCRVARAAAGGKDRHSKVVTARGLRDRRVRLSVPTAIQFYDIQDRLGVDQPSKAIEWLIRAAAAAIDGLPSLDCSFALPTAAGAASPPGAADDAEVSTSDTSKSSVLSLANASADNAAAQQASHAYNGNAAGGGGAFAELILHCSPNDSKPVQQQPTLAYYAAQPPSAHAASALSSFETMPHFSFLQEQPHPAVAFDRGTLQSNAAVAAPLWPTSQQACLLQRFAASPADATGLPFFLGGSAAASPMVPDNSEPRLQLWDFKQERKT

SEQ ID NO: 80 - DNA, 909 bp, Sorghum bicolor
atggaggaggtagcagtagcagcaggcatggagcgcgccgccggctgcaagcgcccgcgcggcgcgctcgacggcggcgggtcggcgtcggcggccgcgtggcggacgtgccgggtggcgcgcgccgcggccggcggcaaggaccggcacagcaaggtggtgacggcgcgggggctgcgggaccgccgcgtccgcctctccgtgcccacggccatccagttctacgacatccaggaccgcctcggcgtcgaccagcccagcaaggccatcgagtggctcatccgcgccgcagccgccgccatcgacggcctcccgtcgctcgactgctccttcgcacttcccaccgccgccggggctgcgtctccgcctggcgctgccgacgacgccgaggtcagcacctccgacaccagcaagagctccgtgctctcgctcgccaacgcctcagctgacaacgccgccgctcagcaagccagtcacgcctacaacggcaacgccgctggcggcggcggcgcgttcgcggagctgatcctgcactgctcccccaacgacagcaagcccgtgcagcagcagccgacgctcgcctactacgccgcgcagcctccgagcgcgcacgccgcgtcggccttgtcgtcgttcgagacgatgcctcacttctccttcctccaggagcagcctcaccccgcagtcgccttcgaccggggcacacttcagtccaatgccgccgttgctgcgccgctgtggccgacgtctcagcaagcgtgcttgctgcagaggttcgccgcttctccggctgatgccaccggactgccgttctttcttggaggcagtgctgctgcttctccgatggtgccagacaattcggagccgaggctgcagctctgggacttcaagcaggaacggaagacctga

SEQ ID NO: 81 - protein, 298 aa, Zea mays
MISGNLNNEELGGGGCTKAEGPGAAGGAGAVVAKSPVLSSSRHWPPSTESRIVRVSRVFGGKDRHSKVRTVKGLRDRRVRLSVPTAIQLYDLQDRLGLSQPSKVVDWLLDAAQDEIDKLPPLQFPPQGQDLVAHLPPSMVAPFANVAADRAAAAAAAANASAAMAEGGKRHCHGGIKGLMGMGLNNSIGLANATMPLAHGLYYTPGESWTNGGNAAVHDHQVSHGGTSPQTVAHHSPFSSLLSLAAPGPQLVFYSPEGGGFAMKEATDHQFAVDSLDHSQGQLSLSSARSFLHPGNQG

SEQ ID NO: 82 - DNA, 897 bp, Zea mays
atgataagcggcaacctcaacaatgaagagctggggggcggcgggtgcaccaaggccgaggggccgggcgcggccggcggcgccggcgccgtcgtcgcaaagagtcccgtgctgtcgagctcccggcactggccgccgtcgacggagtcgaggatcgtgcgcgtgtcgcgggtcttcggcggcaaggaccggcacagcaaggtgaggacggtcaaggggctgcgcgaccggcgcgtgcggctgtccgtgccgacggcgatccagctctacgacctccaggaccggctgggcctcagccagccgagcaaggtggtggactggctgctcgacgccgcgcaggacgagatcgacaagctgcctccgctccagttcccgccgcaagggcaggacctcgtcgcccatctcccgccgtccatggtggcgcccttcgccaacgttgcagccgaccgggccgccgccgccgctgccgccgcgaacgcgtcggccgcgatggccgaaggcggcaaacgccactgccacggaggcatcaaagggctcatgggcatgggcctcaacaattctatcggcctcgccaacgccaccatgccgctggctcacggcttgtactacacgcccggtgagtcttggaccaatggaggcaatgccgccgtccatgatcatcaggtgagccacggcggcacctcgcctcagacggtggctcaccactcgccgttctcgtctctgctgtctcttgctgctccggggccccagctcgtcttctactcgccggaaggcggcggcttcgccatgaaagaagccaccgatcatcagttcgctgtagatagcctcgatcactcgcaaggccagctctcactgagctcagctagaagcttccttcaccccggcaaccagggatga

SEQ ID NO: 83 - protein, 298 aa, Zea mays; identical to SEQ ID NO: 81

SEQ ID NO: 84 - DNA, 897 bp, Zea mays; identical to SEQ ID NO: 82

SEQ ID NO: 85 - protein, 283 aa, Zea mays
MIRGGNHQQAADVEEELARNSRHAAAVATSRQWSAQTESRIVRVSRVFGGKDRHSKVKTVKGLRDRRVRLSVPTAIQLYDLQDRLGLNQPSKVVDWLLNAARHEIDKLPPLQFPPQDLMVGHLTPHNMPLMVHDEKFGHLAAAAALASGGGGGAKVSQQGNVVDVDGTGAHHLVGRFPGGYHRFMELNSALGMVSGAMAPPYNNNYTGEAWNNSGGGVHDSGSPQIVAAAAHHSPFPSLLSLAPRPHQLAFYSSEADQQFQVENLGSQSLSLSSARAYHDQAS

SEQ ID NO: 86 - DNA, 852 bp, Zea mays
atgataaggggcggcaaccaccagcaggcggcggacgtcgaggaggagctagcccggaacagcaggcacgccgccgccgtggcgacgtcgcggcaatggtcggcgcagacggagtcgcgcatcgtgcgcgtctcccgcgtcttcggcggcaaggaccgccacagcaaggtgaagaccgtcaagggcctgcgcgaccggcgggtgcgcctctccgtgccgacggcgatccagctctacgacctgcaggaccgcctcggcctcaaccagcctagcaaggtcgtcgactggctgctcaacgccgcgcgccacgagatcgacaagctgccgccgctgcagttcccgccgcaggacctcatggttgggcacctgacgccccacaacatgccgctgatggtgcacgacgagaagttcggacacctcgccgcggccgcggcgctcgcgagcggcggcggcggcggcgccaaggtcagccagcagggcaacgtcgtcgacgtcgacgggaccggagcccaccacttggtggggaggttccccggcggctaccaccggttcatggagctgaacagcgcgttgggcatggtcagtggcgcaatggcgccgccgtataacaacaactacaccggagaggcatggaataacagcggcggcggcgtgcatgacagcggctcgccgcagatcgtcgccgcagccgctcaccactcgccgttcccatcgttgctctccttggctccacgtccgcatcagctggcgttctactcctcggaagctgatcagcagtttcaggtggagaaccttggctcgcaaagcctgtccttgagctcggcgagggcttaccatgatcaggctagctag

SEQ ID NO: 87 - protein, 283 aa, Zea mays; identical to SEQ ID NO: 85

SEQ ID NO: 88 - DNA, 852 bp, Zea mays; identical to SEQ ID NO: 86

SEQ ID NO: 89 - protein, 283 aa, Zea mays; identical to SEQ ID NO: 85

SEQ ID NO: 90 - DNA, 852 bp, Zea mays; identical to SEQ ID NO: 86

SEQ ID NO: 91 - protein, 275 aa, Zea mays
MISGNLTNEDLGAGRTKAEGPTTAARAVAKSPVLPSSRHWPPSTESRIVRVSRVFGGKDRHSKVRTVKGLRDRRVRLSVPTAIQLYDLQDRLGLSQPSKVVDWLLDAAQHEIDKLPPLQFPPQAAQDLVAHLPPPSMVAPFAADRAEADGDKRHCHAGVKGLMGLNNSIRLVNATMPLAHGLYYTPGESWTGTNADDAAAHDHQTVAHGHSPFSSLLSLAAPGPQLVFYSSEGGGFTMKEEATGYQFPVQNSLDHSQGQLSLSSARSFLRPGNQV

SEQ ID NO: 92 - DNA, 828 bp, Zea mays
atgataagcggcaacctcaccaatgaagacctgggggccgggcgcaccaaggccgaggggccgacgaccgcggccagggccgtcgcgaagagtcccgtgctgccgagctcccggcactggccgccgtcgacggagtccaggatcgtgcgcgtgtcgcgggtcttcggcggcaaggaccggcacagcaaggtgaggacggtcaaggggctgcgcgaccggcgcgtgcggctgtccgtgccgacggcgatacagctctacgacctccaggaccggctgggcctcagccagccgagcaaggtggtggactggctgctcgacgccgcgcagcacgagatcgacaagctgcctccgcttcagttcccaccgcaggcagcgcaggacctcgtcgcccatctgccgccgccatccatggtggcgcccttcgcagccgaccgggccgaggccgacggcgacaaacgccactgccacgccggcgtcaaagggctcatgggcctcaacaattccatccgcctcgtcaacgccaccatgccgctggctcacggcttgtactacacgcccggcgagtcttggaccggtaccaatgccgatgatgccgccgcccatgatcatcagacggtggctcacggccactcgcccttctcatctctgctgtctcttgctgctccggggcctcagctcgtcttctactcgtcggaaggcggcggattcaccatgaaagaagaagccaccggttatcagttcccagtacaaaatagcctcgaccactcgcaaggccagctctcactgagctcagctagaagcttccttcgccccggcaaccaggtatga

SEQ ID NO: 93 - protein, 275 aa, Zea mays; identical to SEQ ID NO: 91

SEQ ID NO: 94 - DNA, 828 bp, Zea mays; identical to SEQ ID NO: 92

SEQ ID NO: 95 - protein, 286 aa, Zea mays
TFASRLRAPPAAAPWAQQHPASRIYRVSRASGGKDRHSKVYTAKGIRDRRVRLSVATAIQFYDLQDRLGYDQPSKAIEWLIRAAAEAIDKLPSLDAAAGFPAHPASAKDHAPPVGDDDDHHHQQQQQLTRSGCSSTSETSKGSVLSLSRSESRVKARERARERNTVARDKDPAANDTTATTSAQAASFTELLTGMAAASAAPAQHKHQLRQSSVSWQPTIVSATTDYIGFAQPRKGGGHAMAHAFASPAPHLGNIASIAMAPAQHFGLASGAVTVASGGGGEPHAE

SEQ ID NO: 96 - DNA, 858 bp, Zea mays
accttcgcctcccgactccgcgcgccgcccgccgccgcgccgtgggcgcagcagcacccggcctcgcggatctaccgcgtctcccgggcctccggcggcaaggaccgccacagcaaggtgtacacggccaagggcatcagggaccgccgcgtccgcctctccgttgccaccgccatccagttctacgacctgcaggaccgcctcggctacgaccagcccagcaaggccatcgagtggctcatcagggccgctgccgaggccatcgacaagctcccctcgctcgacgccgccgcgggcttccccgcgcacccggcctccgccaaggaccacgccccgcctgtcggtgacgacgacgaccaccaccaccagcagcagcagcagctcacgaggtccggatgcagcagcacgtccgagaccagcaagggctccgtcctctccctctcccgctccgagagccgggtcaaggcgcgggagcgcgcaagggagcggaacaccgttgccagggacaaggaccccgccgccaacgacaccaccgccaccacctcggcgcaggcggcgtccttcaccgagctgcttacggggatggccgcggccagcgccgcccccgctcaacacaagcaccagctgcggcagtcgtcggtgtcctggcagccgacgattgtctccgccaccaccgactacatcgggttcgcgcagccccggaagggcggggggcacgcgatggcgcacgccttcgcctcccccgctcctcacctcggcaacattgcgtccatcgccatggccccagcgcagcacttcggcttggccagtggtgccgtcaccgtcgcctccgggggcggcggcgagccgcacgccgag

SEQ ID NO: 97 - protein, 440 aa, Zea mays
MEVEAMGGAGGKRGRAKGAGAGAGAEPEPKRRAMAVAVQPPAAAPWAQQHPASRIYRVSRASGGKDRHSKVYTAKGIRDRRVRLSVATAIQFYDLQDRLGYDQPSKAIEWLIRAAAEAIDKLPSLDAAAGFPAHPASAKDHAPPVGDDDDHHHQQQQQLTRSGCSSTSETSKGSVLSLSRSESRVKARERARERNTVARDKDPAANDTTATTSAQAASFTELLTGMAAASAAPAQHKHQLRQSSVSWQPTIVSATTDYIGFAQPRKGGGHAMAHAFASPAPHLGNIASIAMAPAQHFGLASGAVTVASGGGGEPHAEMTHFSFLQDHFMPVHAAPAPAPASAPAGEYNLDFSMSSGLMGVNTRGALQSNSQPHISGHHHQQQQLQRLSTPLDAPNIPFLYSSAAAAAAAVTTPTTADGQFAAFQLWNGFQHADMKEKGKH

SEQ ID NO: 98 - DNA, 1323 bp, Zea mays
atggaggtggaggccatgggcggcgccgggggcaagcggggcagggccaagggcgcgggcgcgggcgcgggcgccgagccggagccgaagcgacgcgcgatggcggtggccgtgcagccgcccgccgccgcgccgtgggcgcagcagcacccggcctcgcggatctaccgcgtctcccgggcctccggcggcaaggaccgccacagcaaggtgtacacggccaagggcatcagggaccgccgcgtccgcctctccgttgccaccgccatccagttctacgacctgcaggaccgcctcggctacgaccagcccagcaaggccatcgagtggctcatcagggccgctgccgaggccatcgacaagctcccctcgctcgacgccgccgcgggcttccccgcgcacccggcctccgccaaggaccacgccccgcctgtcggtgacgacgacgaccaccaccaccagcagcagcagcagctcacgaggtccggatgcagcagcacgtccgagaccagcaagggctccgtcctctccctctcccgctccgagagccgggtcaaggcgcgggagcgcgcaagggagcggaacaccgttgccagggacaaggaccccgccgccaacgacaccaccgccaccacctcggcgcaggcggcgtccttcaccgagctgcttacggggatggccgcggccagcgccgcccccgctcaacacaagcaccagctgcggcagtcgtcggtgtcctggcagccgacgattgtctccgccaccaccgactacatcgggttcgcgcagccccggaagggcggggggcacgcgatggcgcacgccttcgcctcccccgctcctcacctcggcaacattgcgtccatcgccatggccccagcgcagcacttcggcttggccagtggtgccgtcaccgtcgcctccgggggcggcggcgagccgcacgccgagatgacgcacttctcgttcttgcaggaccatttcatgcccgtccacgcggcaccggcgccggccccggcgtcggctcccgccggagaatacaacctcgacttctccatgtcctcgggtcttatgggtgtcaacactaggggggcccttcagtccaattcgcagccgcatatctccggccaccatcaccagcagcagcagctccagaggctgtctactccactggacgctcccaacatacccttcctgtacagctctgccgccgctgcagctgcggcggtcaccacgcccaccacggccgacggccagttcgccgcgttccagctctggaacgggttccagcacgcagacatgaaggagaagggcaagcactga

SEQ ID NO: 99 - protein, 381 aa, Zea mays
MEAAAVGAGAPGRSQQQGSKRGRGGAVRGSEAEAAWGQPPPPPPPLGPGAGSRIYRVRASGGKDRHSKVYTAKGIRDRRVRLSVPTAIQFYDLQDRLGFDQPSKAIEWLINAAAAAIDKLPELDPAAFAALPAPGDADAAGKDKQQQGSKSAGSSTSETSKGSELSLSRSDSRGGASARDREVTVASTSAQAASFTELLTGVASAGAISAAEHRQSWHHQPNVSAAAAGCVGIAHNGKGAHGLSTHAFSGPAAAKFGSAPPFGLVPAQPFNFTSPIEMPHFSLGQDTLAASSAAAGDYSLGFSMSSGFLGASRGTLQSNSQSNFSGHHHHQLQRLDGPLLFGHVHGAAVAHPASENQLTSSAALQLWDGLRHSGMKEKSKN

SEQ ID NO: 100 - DNA, 1146 bp, Zea mays
atggaggcagcagccgtgggcgccggcgcgcccggccgctcccagcagcagggctccaagcgcggccgcggcggcgccgtgaggggcagcgaggcggaggcggcgtggggccagccgccgccgccgccgccgccgctgggcccgggggcggggtcccggatctaccgcgtgcgggctagcgggggcaaggaccggcacagcaaggtgtacacggccaagggcatccgcgaccgccgcgtgcgcctgtcggtgccaaccgccatccagttctacgacctgcaggaccgcctcggcttcgaccagcccagcaaggccatcgaatggctcatcaacgccgccgccgccgccatcgacaagctcccggagctcgacccggccgccttcgccgccctccccgcgccgggcgacgcggatgccgccggcaaggacaagcagcagcaggggagcaagtccgcgggcagcagcacgtcggagacgagcaagggatccgagctctcgctctcgcgctccgacagccgcgggggcgcctccgcccgggacagggaggtcaccgtggccagcacctcggcgcaggccgcgtccttcaccgagctgctcaccggggtggcgtcggccggcgccattagcgccgccgagcacaggcagtcctggcatcaccagcccaacgtctccgcggccgccgccggctgcgtcggcattgcgcacaacgggaaaggtgcgcacgggctgtcgacgcacgccttttctggccctgccgccgccaagttcggcagcgcgcctccgttcggcctggttccggcgcagcccttcaacttcaccagccccatcgagatgcctcacttctcgctcggccaggacactctggcggcgtcctcggctgccgccggggactacagcctcggtttctccatgtcctcaggtttcttgggtgccagtagggggacccttcagtccaattcgcagtcgaacttctcgggccatcatcaccaccagctccagaggctggatggcccgctcttgtttggtcacgttcacggtgcggcggtggcgcatcccgcatccgagaaccagctcacatcatccgcggcgttacagctgtgggacggtctccggcactccggcatgaaggagaagagcaaaaactga

SEQ ID NO: 101 - protein, 386 aa, Zea mays
MEAAAVGDGAPAGRSHQGAKRARGGAVKGSETEAAAWGQPPPLPPPPGPGAGSRIYRVGASGGKDRHSKVYTTKGIRDRRVRLSAPTAIQFYDLQDRLGFDQPSKAIEWLINAAASAIEKLPELDPAAFAALPAPGGADAAGNDKQQQQGSKSAGSSTSETSKGSELSLSRSDGRGGAAAQDRVVTVASTSAQAASFTELLTGVASAGAISAGEHRQSWHQQPNVSAGAGADRVGIAHNGKGAHGLSTATHAFSGPAAAKFGNAPPFGLVPAQPFDFTIPIEMPHFSLGQDTSAASSSGAGDYSLSFSMSSGFLGANRGTLQSNSQSNFSGHHHQQLQRLDGPLLFGHAHAAAAMHPASENQLTASAALQLWDGLRHSGMKEKGRS

SEQ ID NO: 102 - DNA, 1161 bp, Zea mays
atggaggcagcagccgtgggcgacggcgcgccagcgggccgctcccatcagggcgccaagcgcgcgcgcggcggcgccgtgaagggcagcgagacggaagcggcggcgtggggtcagccgccgccgctcccgccgccgccgggcccgggggcggggtcccggatctaccgcgtgggggcgagcgggggcaaggaccgccacagcaaggtgtacacgaccaagggcatccgcgaccgccgcgtgcgcctgtcggcgcccaccgccatccagttctacgacctgcaggaccgcctcggcttcgaccagcccagcaaggccatcgaatggctcatcaacgccgccgcctccgccatcgagaagctcccggagctcgacccggccgccttcgccgcccttcccgcgccgggcggcgcggatgccgccggcaatgataagcagcagcagcaggggagcaagtccgcgggcagcagcacgtcggagacgagcaaggggtccgagctctcgctctcgcgctccgacggccgtgggggcgccgccgcgcaggacagggtggtcaccgtggccagcacctcggcgcaggccgcgtccttcaccgagctgctcaccggggtggcgtcggccggcgccattagcgccggcgaacataggcagtcgtggcatcagcagcccaacgtctccgccggcgccggcgccgaccgcgtcggcattgcgcacaacgggaaaggcgcgcacgggctgtcgacggcgacgcacgccttttctggccccgccgccgccaagtttggcaatgcgcctccgttcggcctggtgccggcacagcctttcgacttcaccatccccatcgagatgcctcacttctcgctcggtcaggacacttcggcggcgtcctcgtctggcgccggggactacagcctcagtttttccatgtcctcgggtttcttgggtgccaatagggggacccttcagtccaattcgcagtcgaacttctcaggccatcatcaccagcagctccagaggctggacggcccgctcttgtttggccacgcccacgccgcggcggcgatgcatcccgcatccgagaaccagctgaccgcatcggcggcgttacagctctgggacggactccggcactccggcatgaaggagaagggcaggagctga

SEQ ID NO: 103 - DNA, 22 bp, Artificial
  primer
atgaatatcgtctcttggaaag

SEQ ID NO: 104 - DNA, 23 bp, Artificial
  primer
tcacatatggtgatcacttcctc

SEQ ID NO: 105 - DNA, 22 bp, Artificial
  primer
atgagatcaggagaatgtgatg

SEQ ID NO: 106 - DNA, 23 bp, Artificial
  primer
tcaagaatctgattcattatcgc

SEQ ID NO: 107 - DNA, 22 bp, Artificial
  primer
atgggaataaaaaaagaagatc

SEQ ID NO: 108 - DNA, 23 bp, Artificial
  primer
ctactcgatatggtctggttgtg

(19) United States
(12) Patent Application Publication
(10) Pub. No.: US 2013/0019340 A1
(43) Pub. Date: Jan. 17, 2013

(54) PLANT RESISTANT TO ENVIRONMENTAL STRESS AND METHOD FOR PRODUCING THE SAME

(71) Applicants: Kaoru Maruyama, Yokohama-Shi (JP); Kazuo Shinozaki, Kanagawa (JP)

(72) Inventors: Kaoru Maruyama, Yokohama-Shi (JP); Kazuo Shinozaki, Kanagawa (JP)

(73) Assignee: RIKEN, Wako-shi, Saitama (JP)

(21) Appl. No.: 13/635,390

(22) PCT Filed: Mar. 1, 2011
(86) PCT No.: PCT/JP2011/055171

§ 371 Date: Sep. 14, 2012

(30) Foreign Application Priority Data
Mar. 16, 2010 (JP) 2010-059022

(51) Int. Cl. C12N 15/82

(52) U.S. Cl. 800/278

(57) ABSTRACT

An object of the present invention is to provide a plant having environmental stress resistance. The present invention provides a transgenic plant having environmental stress resistance, in which an exogenous DNA encoding a transcription factor selected from the group consisting of transcription factors from Arabidopsis thaliana TCP13, TCP5 and TCP17, and homologous transcription factors thereof from other plants is overexpressed, and a method for producing such a plant.

### TECHNICAL FIELD

The present invention relates to a plant resistant to environmental stress that comprises a TCP transcription factor gene in a manner that allows overexpression and a method for producing the same.

### BACKGROUND ART

The global population has been increasing with environmental changes. It is expected that global issues such as desertification and food shortages resulting therefrom would become serious in the near future. As a solution to the problem of how plants (mainly cereals) are made adjusted to environmental changes, development of plants resistant to a variety of environmental stresses has been actively conducted.

To date, a variety of plants resistant to stresses by environmental conditions such as high temperature, low temperature, drought, high salinity, disease and pesticide have been reported.

There are many examples of such environmental stress-resistant plants in which a particular gene is overexpressed in response to the environmental stress to confer stress resistance thereon. Known examples of such a gene include a gene encoding the chloroplast-localizing protein (Patent Literature 1), the GST gene or the glutathione peroxidase gene (Patent Literature 2), the sHSP17.7 gene (Patent Literature 3), the betaine synthetase gene (Patent Literature 4) and the ascorbate peroxidase gene (Patent Literature 5).

Further, stress-inducible transcription factors also can confer environmental stress resistance on plants. DREB and AREB have been known as major transcription factors, and have been used for production of plants resistant to environmental stress.

As DREB, DREB1A, DREB1B, DREB1C, DREB2A and DREB2B are known, for example. When such a transcription factor is overexpressed in a plant, the plant becomes stress-resistant (Non-Patent Literature 1, Patent Literature 6, and Patent Literature 7). It also has been reported that complex environmental stress resistance can be attained in plants belonging to the family Gramineae by modifying DREB2A (Patent Literature 8).

AREB is a transcription factor that binds to a promoter (ABRE) of a gene controlled by abscisic acid (ABA). ABA is synthesized under water-deprived conditions and plays an important role in response to drought stress. There are many known genes induced by drought stress, and many of them are activated by ABA (Patent Literature 9). As AREB, Arabidopsis thaliana AREB 1, AREB2 and AREB3 are known, for example.

Hitherto, DREB and AREB have been mainly used for production of a plant resistant to environmental stress by introducing a stress-inducible transcription factor. They have been identified as transcription factors that positively control genes whose expression is increased under environmental stress. Although more than 10 years have passed since the discovery of these genes, no novel transcription factor that confers environmental stress resistance to levels comparable to those by the above transcription factors has been found.

Many genes are involved in the downstream pathway of a transcription factor. Thus, it is considered that if a transcription factor other than DREB and AREB that is involved in environmental stress response in a plant is found, it would become possible to efficiently confer resistance to various environmental stresses on plants.
(Patent Literature 1) JP Patent Publication (Kokai) No. 2007-222129 A
(Patent Literature 2) JP Patent Publication (Kokai) No. 2007-166986 A
(Patent Literature 3) JP Patent Publication (Kokai) No. 2006-034252 A
(Patent Literature 4) JP Patent Publication (Kokai) No. 2003-143988 A
(Patent Literature 5) JP Patent Publication (Kokai) No. 2003-009692 A
(Patent Literature 6) JP Patent Publication (Kokai) No. 2003-219882 A
(Patent Literature 7) JP Patent Publication (Kokai) No. 2003-219891 A
(Patent Literature 8) JP Patent Publication (Kohyo) No. 2008-505603 A
(Patent Literature 9) JP Patent Publication (Kokai) No. 2007-124925 A
(Non-Patent Literature 1) Lie Q. et al., The Plant Cell, Vol. 10, 1391-1406, 1998

### DISCLOSURE OF INVENTION

An object of the present invention is to confer environmental stress resistance on a plant utilizing a transcription factor other than DREB and AREB.

In the past, research on transcription factors that confer environmental stress resistance focused on DREB and AREB. One reason for the little progress in the discovery of novel transcription factors is that the research focused only on analysis of a factor controlling a group of genes whose expression is increased under stress.

The present inventors analyzed a group of genes whose expression is decreased under environmental stress in order to isolate a transcriptional repressor that controls such genes. As a result, it was clarified that expression of a family of genes involved in protein synthesis is commonly decreased in response to environmental stress and that the promoter sequences of the genes have a common cis sequence, namely Up1 (GGCCCAWW)/Site II (GGNCCC). Since it has been revealed that TCP transcription factors bind to the Site II sequence, the present inventors attempted to isolate a TCP transcription factor whose expression is increased under drought stress. As a result of analysis of expression of the TCP gene family in Arabidopsis thaliana under environmental stress, TCP13 and a homolog thereof were isolated as environmental stress-inducible TCP transcription factors. In fact, when the transcription factor was overexpressed in Arabidopsis thaliana plants, remarkable increase in environmental stress resistance was observed.

Specifically, the present invention includes the following inventions.

(1) A transgenic plant having environmental stress resistance, which comprises exogenous DNA encoding a TCP transcription factor selected from the group consisting of transcription factors from Arabidopsis thaliana TCP13, TCP5 and TCP17, and homologous transcription factors thereof from other plants, and in which the DNA is overexpressed.

(2) The transgenic plant according to (1), wherein the transcription factor TCP13 or the homologous transcription factor thereof comprises the following protein (a) or (b):

(a) a protein consisting of the amino acid sequence shown in SEQ ID NO: 1, 3, 13, 15, 17, 19, 21, 23, 33, 35, 37, 39, 43, 55, 57, 59, 61, 63, 67, 69, 75, 77, 79, 95, 97, 99 or 101; or

(b) a protein consisting of an amino acid sequence having at least 70% identity to the amino acid sequence as recited in (a) and being capable of conferring environmental stress resistance on a plant.

(3) The transgenic plant according to (1), wherein the transcription factor TCP5 or TCP17, or the homologous transcription factor thereof comprises the following protein (c) or (d):

(c) a protein consisting of an amino acid sequence shown in SEQ ID NO: 5, 7, 9, 11, 13, 15, 17, 25, 27, 29, 31, 33, 35, 37, 39, 41, 45, 47, 49, 51, 53, 55, 57, 65, 67, 69, 71, 73, 75, 77, 79, 81, 83, 85, 87, 89, 91, 93, 95, 97, 99 or 101; or

(d) a protein consisting of an amino acid sequence having at least 70% identity to the amino acid sequence as recited in (c) and being capable of conferring environmental stress resistance on a plant.

(4) The transgenic plant according to (1) or (2), wherein the DNA encoding the transcription factor TCP13 or the homologous transcription factor thereof is any of the following DNAs (e) to (g):

(e) DNA consisting of the nucleotide sequence shown in SEQ ID NO: 2, 4, 14, 16, 18, 20, 22, 24, 34, 36, 38, 40, 44, 56, 58, 60, 62, 64, 68, 70, 76, 78, 80, 96, 98, 100 or 102;

(f) DNA consisting of a nucleotide sequence having at least 70% identity to the nucleotide sequence as recited in (e) and encoding a protein capable of conferring environmental stress resistance on a plant;

(g) DNA that hybridizes under stringent conditions to DNA consisting of a nucleotide sequence complementary to DNA consisting of the nucleotide sequence as recited in (e) and encodes a protein capable of conferring environmental stress resistance on a plant.

(5) The transgenic plant according to (1) or (3), wherein the DNA encoding the transcription factor TCP5 or TCP17, or the homologous transcription factor thereof is any of the following DNAs (h) to (j):

(h) DNA consisting of the nucleotide sequence shown in SEQ ID NO: 6, 8, 10, 12, 14, 16, 18, 26, 28, 30, 32, 34, 36, 38, 40, 42, 46, 48, 50, 52, 54, 56, 58, 66, 68, 70, 72, 74, 76, 78, 80, 82, 84, 86, 88, 90, 92, 94, 96, 98, 100 or 102;

(i) DNA consisting of a nucleotide sequence having at least 70% identity to the nucleotide sequence as recited in (h) and encoding a protein capable of conferring environmental stress resistance on a plant;

(j) DNA that hybridizes under stringent conditions to DNA consisting of a nucleotide sequence complementary to DNA consisting of the nucleotide sequence as recited in (h) and encodes a protein capable of conferring environmental stress resistance on a plant.

(6) The transgenic plant according to any one of (1) to (5), wherein the environmental stress resistance is salt stress resistance or drought stress resistance.

(7) A progeny of the transgenic plant of any one of (1) to (6).

(8) A cell, tissue, or organ from the transgenic plant of any one of (1) to (6) or the progeny of (7).

(9) A seed from the transgenic plant of any one of (1) to (6) or the progeny of (7).

(10) A method of producing a transgenic plant of any one of (1) to (6), comprising:

introducing an exogenous DNA encoding a transcription factor selected from the group consisting of transcription factors from Arabidopsis thaliana TCP13, TCP5 and TCP17, and homologous transcription factors thereof from other plants into a cell or tissue of a plant in an expressible manner; and

regenerating a plant from the cell or tissue.

According to the present invention, resistance to stress by environmental conditions such as drought or high salinity can be conferred on a plant by overexpressing DNA encoding an environmental stress-inducible TCP transcription factor in the plant.

### BRIEF DESCRIPTION OF DRAWINGS

FIG. 1 shows overexpression of the TCP13 gene in T2 plants of transformed Arabidopsis thaliana strains OX1, OX2, OX3, OX10, OX14, OX20, OX21 and OX28 (A) and the growth status of the transformed strains (B).

FIG. 2 shows the growth status of transformed Arabidopsis thaliana strains OX3, OX10, OX14 and OX20 under high salinity (225 mM NaCl, Day 12) (A), and a graph showing the chlorophyll content in each of transformed Arabidopsis thaliana strains OX3, OX10, OX14 and OX20 grown under the same high salinity (B).

FIG. 3 shows the growth status (A) and a graph showing the survival rates (B) of the transformed Arabidopsis thaliana strains OX3 and OX28 after drought stress treatment.

FIG. 4 shows the growth status of transformed Arabidopsis thaliana strains 5OX-10, 5OX-16, 5OX-17 and 5OX-23 under high salinity (225 mM NaCl, Day 12) (A), a graph showing the chlorophyll content in each of the transformed Arabidopsis thaliana strains 5OX-10, 5OX-16, 5OX-17 and 5OX-23 grown under the same high salinity (B), the growth status of the transformed Arabidopsis thaliana strains 17OX-3, 17OX-9, 17OX-14 and 17OX-15 under high salinity (225 mM NaCl, Day 12) (C), and a graph showing the chlorophyll content in each of the transformed Arabidopsis thaliana strains 17OX-3, 17OX-9, 17OX-14 and 17OX-15 grown under the same high salinity (D).

The present application claims priority to Japanese Patent Application No. 2010-059022 filed on Mar. 16, 2010, and the contents of the patent application are herein incorporated by reference.

### BEST MODES FOR CARRYING OUT THE INVENTION

Hereinafter, the present invention is described in detail.

### <Definition>

The terms used herein include the following meanings. In addition, other terms used herein include the meanings commonly used in the art.

The term “TCP transcription factor” is not intended to be limited to transcription factors from Arabidopsis thaliana TCP13, TCP5 and TCP17, and it also includes homologs of such TCP transcription factors from any plant other than Arabidopsis thaliana. Thus, a “TCP transcription factor” may be referred to as a TCP13-, TCP5- or TCP17-related transcription factor. TCP13, TCP5 and TCP17 have sequences very similar to one another, and TCP5 and TCP17 have sequences even more similar to each other. Accordingly, TCP13, TCP5 and TCP17 are considered to be closely related to one another in terms of the biological function.

TCP is an abbreviated designation for Teosinte branched 1 (tb1) from maize, Cycloidea (cyc) from Antirrhinum, and PCF proteins from rice. TCP refers to a plant-specific family of transcription factors each containing the bHLH (basic helix-loop-helix) domain. TCP proteins are classified into two subfamilies, namely class I and class II. There are 24 types of TCP members in Arabidopsis thaliana. A promoter targeted by TCP transcription factors is presumed to be Up1 (GGCCCAWW)/Site II (GGNCCC). As a result of research on Arabidopsis thaliana, it is known that TCP members function in morphogenesis, circadian clock, and seed germination (Palatnik et al., Nature 425: 257-263 (2003); A-Martinez et al., Plant Cell 19:458-72 (2007); Koyama et al., 2007 Plant Cell 19:473-484 (2007); Tatematsu et al., The Plant Journal 53:42-52 (2008); Paz et al., Science 323:1481-1485 (2009); Hervoe et al., Plant Physiol. 149:1462-77 (2009)).

Of the aforementioned TCP members, the TCP according to the present invention is a TCP13-, TCP5- or TCP17-related transcription factor. Phylogenetic analysis revealed that TCP13, TCP5 and TCP17 belong to class II and are closely related functionally associated transcription factors (Mar Martin-Trillo and Pilar Cubas, Trends in Plant Science, 15:31-39, 2010, Elsevier Science).

The expression “homologous transcription factors from other plants” refers to proteins that are TCP transcription factors having biological functions equivalent to those of TCP13, TCP5 or TCP17 described above in plant species other than Arabidopsis thaliana and can confer environmental stress resistance on a plant.

The “other plants” as used herein include, but are not limited to: plants belonging to the families Brassicaceae, Gramineae, Leguminosae, Solanaceae, Liliaceae, Fagaceae, Chenopodiaceae, Myrtaceae, Salicaceae and Arecaceae; and mosses. More specific examples include Arabidopsis thaliana, Brassica napus (rapeseed), broccoli, radish, cauliflower, cabbage, Chinese white cabbage, rice, wheat, barley, maize, Brachypodium distachyon, soybean, Lotus japonicus, Medicago truncatula, tomato, eggplant, potato, green onion, onion, garlic, spinach, sugar cane, eucalyptus, poplar, oil palm, horseradish, leek and Physcomitrella patens.

The term “environmental stress” as used herein with respect to the expression “environmental stress resistance” includes at least salt stress and drought stress. Other examples include low temperature stress, high temperature stress, disease stress and pesticide stress. Preferable environmental stress is salt stress, drought stress, or low temperature stress.

When plants are exposed to environmental stress, they respond to stress by increasing expression levels of a variety of genes and products thereof. Suppression of the growth rates of plants is also an important mechanism for adaptation to the environment. Environmental stress resistance according to the present invention can be acquired by overexpressing a TCP transcription factor in a plant so as to control factor(s) involved in the growth of the plant.

The term “identity” as used herein with respect to amino acid or nucleotide sequences refers to a percentage (%) of the number of identical amino acid residues or nucleotides relative to the total number of amino acid residues or nucleotides including the number of gaps observed when two sequences are aligned with or without the introduction of gaps. Determination of identity between sequences can be performed by utilizing known algorithms, such as BLAST (e.g., BLASTN, BLASTP, or BLASTX) or FASTA (Nucl. Acids Res., 25:3389-3402, 1997; Proc. Natl. Acad. Sci. USA, 85: 2444-2448, 1988).

“DNA” as used herein includes a gene, genomic DNA and cDNA.

### <TCP Transcription Factor>

A TCP transcription factor or DNA encoding the transcription factor used in the present invention includes a transcription factor selected from the group consisting of transcription factors from Arabidopsis thaliana TCP13, TCP5 and TCP17, and homologous transcription factors thereof from other plants, or DNA encoding such a transcription factor.

DNA encoding a TCP transcription factor used in the present invention is introduced exogenously into a plant in an expressible manner so that the DNA is overexpressed in the plant. TCP transcription factors include all proteins that function in plants in which such proteins are expressed so as to confer environmental stress resistance on the plants. Such proteins include not only naturally occurring TCP transcription factors from plants but also mutants thereof. Such mutants may be spontaneously generated mutants or mutants produced by artificial mutagenesis, as long as they can confer environmental stress resistance on plants.

Such a mutant comprises an amino acid or nucleotide sequence derived from the amino acid or nucleotide sequence of a naturally occurring TCP transcription factor or DNA encoding the transcription factor by deletion, substitution, or addition of one or a plurality of (preferably one or several) amino acids or nucleotides. The term “a plurality of” as used herein for amino acids refers to an integer of, for example, 2 to 100, preferably 2 to 70, more preferably 2 to 50, and further preferably 2 to 30. For nucleotides, it refers to an integer of, for example, 2 to 300, preferably 2 to 200, more preferably 2 to 20, and further preferably 2 to 10. The term “several” as used herein refers to an integer of 2 to 10. Substitution includes conservative amino acid substitution.

Conservative amino acid substitution means substitution between amino acids having similar properties, for example, in terms of structural, electrical, polar, or hydrophobic properties or the like. Such properties can be classified based on, for example, similarity in amino acid side chains. Examples of amino acids having basic side chains include lysine, arginine and histidine. Examples of amino acids having acidic side chains include aspartic acid and glutamic acid. Examples of amino acids having uncharged polar side chains include glycine, asparagine, glutamine, serine, threonine, tyrosine and cysteine. Examples of amino acids having hydrophobic side chains include alanine, valine, leucine, isoleucine, proline, phenylalanine and methionine. Examples of amino acids having branched side chains include threonine, valine, leucine and isoleucine. Examples of amino acids having aromatic side chains include tyrosine, tryptophan, phenylalanine and histidine.

Alternatively, if a mutant is a nucleic acid, it comprises a nucleotide sequence that hybridizes under stringent conditions to DNA consisting of a sequence complementary to a nucleotide sequence encoding a naturally occurring TCP transcription factor. The term “stringent conditions” includes, for example, the condition of hybridization carried out at about 42° C. to 55° C. in the presence of 2× to 6×SSC, followed by washing once or several times at 50° C. to 65° C. in the presence of 0.1× to 1×SSC and 0.1% to 0.2% SDS. Since such conditions vary depending on the GC content of the nucleic acid as a template, ionic strength, temperature, and other factors, the conditions are not limited to those specifically described above. 1×SSC is composed of 0.15 M NaCl and 0.015 M sodium citrate (pH 7.0). In general, stringent conditions are set so that the temperature is lower by about 5° C. than the melting temperature (Tm) of a given sequence at the designated ion intensity and pH. Tm refers to a temperature at which 50% of the probes complementary to a template sequence hybridize to the template sequence at equilibrium.

Alternatively, a mutant comprises an amino acid or nucleotide sequence having at least 70% to 85%, more preferably at least 90%, further preferably at least 95%, and most preferably at least 98% identity to the amino acid or nucleotide sequence of a naturally occurring TCP transcription factor or DNA encoding the same.

Non-limiting specific examples of a TCP transcription factor and DNA encoding the same are described below.

(1) Transcription factor TCP13 or a homologous transcription factor thereof:

(a) a protein consisting of the amino acid sequence shown in SEQ ID NO: 1, 3, 13, 15, 17, 19, 21, 23, 33, 35, 37, 39, 43, 55, 57, 59, 61, 63, 67, 69, 75, 77, 79, 95, 97, 99 or 101; or

(b) a protein consisting of an amino acid sequence having at least 70% identity to the amino acid sequence as recited in (a) and being capable of conferring environmental stress resistance on a plant.

(2) Transcription factor TCP5 or TCP17, or a homologous transcription factor thereof:

(c) a protein consisting of the amino acid sequence shown in SEQ ID NO: 5, 7, 9, 11, 13, 15, 17, 25, 27, 29, 31, 33, 35, 37, 39, 41, 45, 47, 49, 51, 53, 55, 57, 65, 67, 69, 71, 73, 75, 77, 79, 81, 83, 85, 87, 89, 91, 93, 95, 97, 99 or 101; or

(d) a protein consisting of an amino acid sequence having at least 70% identity to the amino acid sequence as recited in (c) and being capable of conferring environmental stress resistance on a plant.

Transcription factors TCP5 and TCP17 have sequences that are very similar to each other.

(3) DNA encoding the transcription factor TCP13 or a homologous transcription factor thereof:

(e) DNA consisting of the nucleotide sequence shown in SEQ ID NO: 2, 4, 14, 16, 18, 20, 22, 24, 34, 36, 38, 40, 44, 56, 58, 60, 62, 64, 68, 70, 76, 78, 80, 96, 98, 100 or 102;

(f) DNA consisting of a nucleotide sequence having at least 70% identity to the nucleotide sequence as recited in (e) and encoding a protein capable of conferring environmental stress resistance on a plant; or

(g) DNA that hybridizes under stringent conditions to DNA consisting of a nucleotide sequence complementary to DNA consisting of the nucleotide sequence as recited in (e) and encodes a protein capable of conferring environmental stress resistance on a plant.

(4) DNA encoding the transcription factor TCP5 or TCP17, or a homologous transcription factor thereof:

(h) DNA consisting of the nucleotide sequence shown in SEQ ID NO: 6, 8, 10, 12, 14, 16, 18, 26, 28, 30, 32, 34, 36, 38, 40, 42, 46, 48, 50, 52, 54, 56, 58, 66, 68, 70, 72, 74, 76, 78, 80, 82, 84, 86, 88, 90, 92, 94, 96, 98, 100 or 102;

(i) DNA consisting of a nucleotide sequence having at least 70% identity to the nucleotide sequence as recited in (h) and encoding a protein capable of conferring environmental stress resistance on a plant; or

(j) DNA that hybridizes under stringent conditions to DNA consisting of the nucleotide sequence complementary to DNA consisting of the nucleotide sequence as recited in (h) and encodes a protein capable of conferring environmental stress resistance on a plant.

Table 1 shows the origins (organism names), GenBank accession nos., and SEQ ID NOs of the above transcription factors.

TABLE 1

| | | SEQ ID: NO | |
|---|---|---|---|
| | GenBank | Amino acid | Nucleotide |
| Organism name | Accession No. | sequence | sequence |
| Arabidopsis | AT3G02150.2(TCP13) | 1 | 2 |
| thaliana | AT3G02150.1(TCP13) | 3 | 4 |
| | AT5G60970(TCP5) | 5 | 6 |
| | AT5G08070(TCP17) | 7 | 8 |
| Brachypodium | Bradi2g50690.1 | 9 | 10 |
| distachyon | Bradi2g20060.1 | 11 | 12 |
| | Bradi4g01550.1 | 13 | 14 |
| | Bradi1g06460.1 | 15 | 16 |
| | Bradi1g58450.1 | 17 | 18 |
| Glycine max | Glyma08g27150.1 | 19 | 20 |
| | Glyma19g03810.1 | 21 | 22 |
| | Glyma18g50370.1 | 23 | 24 |
| | Glyma17g08760.1 | 25 | 26 |
| | Glyma06g22240.1 | 27 | 28 |
| | Glyma04g32340.1 | 29 | 30 |
| | Glyma05g00300.1 | 31 | 32 |
| | Glyma15g09910.1 | 33 | 34 |
| | Glyma13g29160.1 | 35 | 36 |
| | Glyma05g27370.1 | 37 | 38 |
| | Glyma08g10350.1 | 39 | 40 |
| Medicago | Medtr3g036490.1 | 41 | 42 |
| truncatula | | | |
| Lotus japonicus | LjSGA_022391.1 | 43 | 44 |
| | chr4.CM0500.330.nd | 45 | 46 |
| Oryza sativa | Os01g55750.1 | 47 | 48 |
| | Os01g55750.3 | 49 | 50 |
| | Os01g55750.2 | 51 | 52 |
| | Os05g43760.1 | 53 | 54 |
| | Os07g05720.1 | 55 | 56 |
| | Os03g57190.1 | 57 | 58 |
| Physcomitrella | 97688 | 59 | 60 |
| patens | | | |
| Populus | POPTR_0004s11510.1 | 61 | 62 |
| trichocarpa | POPTR_0017s13050.1 | 63 | 64 |
| | POPTR_0015s06980.1 | 65 | 66 |
| | POPTR_0004s06440.1 | 67 | 68 |
| | POPTR_0011s02350.1 | 69 | 70 |
| Sorghum bicolor | Sb09g025340.1 | 71 | 72 |
| | Sb03g035350.1 | 73 | 74 |
| | Sb02g003070.1 | 75 | 76 |
| | Sb01g006020.1 | 77 | 78 |
| | Sb08g021690.1 | 79 | 80 |
| Zea mays | GRMZM2G120151_T02 | 81 | 82 |
| | GRMZM2G120151_T01 | 83 | 84 |
| | GRMZM2G166946_T03 | 85 | 86 |
| | GRMZM2G166946_T01 | 87 | 88 |
| | GRMZM2G166946_T02 | 89 | 90 |
| | GRMZM2G035944_T01 | 91 | 92 |
| | GRMZM2G035944_T02 | 93 | 94 |
| | GRMZM2G020805_T02 | 95 | 96 |
| | GRMZM2G020805_T01 | 97 | 98 |
| | GRMZM2G089361_T01 | 99 | 100 |
| | GRMZM2G115516_T01 | 101 | 102 |

The TCP transcription factor and DNA encoding the same of the present invention can be amplified from a genomic library or cDNA library from an organ or tissue of a plant in which the DNA is expressed using a general cloning method, polymerase chain reaction (PCR) method or the like.

Gene recombination techniques, including cloning method and PCR, are described in, for example, Sambrook et al., Molecular Cloning A Laboratory Manual, 1989, Cold Spring Harbor Laboratory Press, and Ausubel et al., Current Protocols in Molecular Biology, 1994, John Wiley & Sons.

Examples of plant organs include root, stem, leaf, petal, seed and fruit. Examples of plant tissues include epidermis, phloem, parenchyma, xylem, vascular bundle, palisade tissue and spongy tissue.

### <Transformation of Plants>

In the present invention, a plant, or a plant cell or tissue, is transformed with a vector containing DNA encoding the aforementioned TCP transcription factor in order to produce a plant resistant to environmental stress. A preferable example of such vector is a binary vector.

A binary vector comprises two border sequences of about 25 by (i.e., a right border (RB) sequence and a left border (LB) sequence) from Agrobacterium T-DNA, and exogenous DNA is inserted between the border sequences. A promoter is ligated to the 5′ side of the exogenous DNA.

Examples of promoters include cauliflower mosaic virus (CaMV) 35S promoter (Jefferson, R. A. et al.: The EMBO J 6:3901-3907 (1987)), nopaline synthase gene promoter (Christensen, A. H. et al.: Plant Mol. Biol. 18:675-689 (1992)), maize ubiquitin promoter, octopine synthase gene promoter, and rice actin promoter. Other examples of promoters include rd29A gene promoter, rd29B gene promoter, rd17 gene promoter, rd22 gene promoter, cor6.6 gene promoter, cor15a gene promoter, erd1 gene promoter, and kin1 gene promoter.

Further, a terminator (e.g., nopaline synthase gene terminator or terminator from cauliflower mosaic virus) is inserted at the 3′ end of exogenous DNA.

A selection marker that is necessary for selecting a transformed cell can be further inserted into a vector. Examples of selection markers include drug resistance genes, such as kanamycin resistance gene (NPTII), hygromycin resistance gene (htp), and bialaphos resistance gene (bar).

Examples of binary vectors include, but are not limited to, pBI-series such as pBI101, pBI101.2, pBI101.3, pBI121, pBI221, pBE2113Not, pBI2113Not, pBI2113, pGA482, pGAH, and pBIG. Examples of other vectors include intermediate plasmids such as pLGV23Neo, pNCAT, and pMON200, as well as pH35GS which comprises the GATEWAY cassette (Kubo et al., 2005. Genes & Dev. 19: 1855-1860).

An example of a transformation method for introducing the vector into a plant is a method using Agrobacterium. In addition, the gene gun method, the electroporation method, the virus vector method, the floral dip method, and the leaf disc method can be used for the introduction. Plant transformation techniques and tissue culture techniques are described in, for example, Ko Shimamoto, Kiyotaka Okada (ed.), Shokubutsu Saibou Kougaku Series 15, Model Shokubutsu No Jikken Protocol, Idengakuteki Shuhou Kara Genome Kaiseki Made (Plant Cell Technology Series 15, Experimental Protocol for Model Plants, From Genetic Technique to Genome Analysis), Shujunsha, (Tokyo, Japan), 2001.

According to a method utilizing the binary vector-Agrobacterium system as a vector, plant-derived cells, tissues, or calluses are prepared, such materials are infected with Agrobacterium, and DNA encoding a TCP transcription factor of the present invention is introduced into the plant cells. Particularly in the case of transformation of a monocotyledonous plant, it is desirable to add a phenolic compound (e.g., acetosyringon) to a medium. An Agrobacterium tumefaciens strain, such as C58, LBA4404, EHA101, EHA105, or C58C1RifR, can be used as Agrobacterium.

For a medium used for transformation, for example, 1% to 5% of saccharides, such as maltose, sucrose, glucose or sorbitol, and 0.2% to 1% of polysaccharide solidification agents, such as agar, agarose or gellan gum (Gelrite®), can be added to a basal medium (i.e., a plant culture medium, such as MS medium, B5 medium, DKN medium or Linsmaier-Skoog medium). Casamino acid, auxins or cytokinins (e.g., abscisic acid, kinetin, 2,4-D, indoleacetic acid and indolebutyric acid), antibiotics (e.g., kanamycin, hygromycin and carbenicillin), acetosyringone, and the like can be adequately added to a medium. The preferable pH of the medium is 5 to 6, for example, pH 5.5 to 5.8.

Specifically, a suspension of Agrobacterium cells is prepared by culture in the dark at about 25° C. for about 4 days, the plant calluses or tissues (e.g., laminae, roots, stem segments or meristems) are soaked in the cell suspension for several minutes, moisture is removed therefrom, and the cells are then sown on a solid medium to conduct coculture. A callus is a mass of plant cells, and it can be induced from a plant tissue segment, a mature seed or the like using a callus induction medium. A transformed callus or tissue segment is selected with the aid of a selection marker. In case of callus, the callus can then be redifferentiated into a seedling in a redifferentiation medium. In case of plant segment, a callus may be induced from the plant segment, and redifferentiated into a seedling. Alternatively, a protoplast may be prepared from the plant segment, subjected to callus culture, and then redifferentiated into a seedling. The thus-obtained seedling is transferred to soil after rooting, and regenerated into a plant body.

For transformation using the floral dip method, for example, as described in Clough and Bent et al., Plant J. 16, 735-743, 1998, a suspension of Agrobacterium cells is prepared by culture in the dark at about 25° C. for about 4 days, flower buds of a plant host to be transformed (which had been grown to develop premature flower buds) are soaked in the cell suspension for about 10 seconds, and the resultant is covered to maintain humidity overnight. The cover is removed on the following day, the plant is allowed to grow, and seeds are then harvested. Transformed plant individuals can be selected by further sowing the harvested seeds on a solid medium to which an adequate selection marker, such as an antibiotic, has been added. Further, the thus-selected individuals can be transferred to soil and grown to obtain the progeny seeds of transformed (or transgenic) plants.

Examples of host plants that can be used for transformation in the present invention include: plants such as dicotyledonous plants, monocotyledonous plants, gymnosperms, and trees. Specific examples include plants belonging to the families Brassicaceae, Gramineae, Leguminosae, Solanaceae, Liliaceae, Chenopodiaceae, Fagaceae, Myrtaceae, Salicaceae and Arecaceae, and mosses. More specific examples include Arabidopsis thaliana, Brassica napus (rapeseed), broccoli, radish, cauliflower, cabbage, Chinese white cabbage, rice, wheat, barley, maize, Brachypodium distachyon, soybean, Lotus japonicus, Medicago truncatula, tomato, eggplant, potato, green onion, onion, garlic, spinach, sugar cane, eucalyptus, poplar, oil palm, horseradish, leek and Physcomitrella patens.

For transformation, the whole body of the aforementioned plant, as well as an organ (e.g., leaf, petal, stem, root, rhizome or seed), a tissue (e.g., epidermis, phloem, parenchyma, xylem or vascular bundle), and a cultured plant cell (e.g., callus) of the aforementioned plant can be used.

A transformed tissue, shoot, hairy root, or the like can be regenerated into a plant body by administering an adequate plant hormone, such as auxin, cytokinin, gibberellin, abscisic acid, ethylene or brassinolide according to, for example, a known plant tissue culture method.

Whether or not DNA of interest has been introduced into a transgenic plant can be confirmed using the PCR method, the Southern hybridization method, the Northern hybridization method, the Western blot method or the like.

The transgenic plant produced as described above has environmental stress resistance. Environmental stress resistance includes at least salt stress and drought stress. Other examples include low temperature stress, high temperature stress, disease stress and pesticide stress. Preferable environmental stress is salt stress, drought stress or low temperature stress.

Environmental stress resistance of the transgenic plant of the present invention can be evaluated by, for example, planting a transgenic plant in a pot containing soil comprising vermiculite, pearlite, or the like and examining the survival of the plant when exposing the plant to a variety of environmental stresses.

Drought stress resistance is assessed by, for example, stopping water supply for 6 days during cultivation and observing the growth status.

Salt stress resistance is assessed by, for example, observing whether or not a plant can be grown under high salinity (200 mM or more).

Low temperature stress resistance is assessed by treating a plant at −6° C. for 2 to 3 days and then observing the growth status under ordinary temperature conditions.

The present invention is hereafter described in greater detail with reference to the following examples, although the present invention is not limited thereto.

### EXAMPLE 1

### Production of TCP13-Overexpressing Plants

PCR reaction was performed using reverse-transcribed DNA prepared from total RNA of Arabidopsis thaliana that had been subjected to drought stress treatment as a template and the following PCR primers: TCP13F (ATGAATATCGTCTCTTGGAAAG: SEQ ID NO: 103); and TCP13R (TCACATATGGTGATCACTTCCTC: SEQ ID NO: 104). The following were used as PCR conditions with reference to the manufacturer's protocol for Phusion High-Fidelity DNA Polymerase (http://www.neb.com/nebecomm/): 98° C. for 30 sec→30 cycles×98° C. for 10 sec, 58° C. for 10 sec, and 72° C. for 30 sec→72° C. for 7 min→4° C. (for cooling). The amplification product was cloned by introducing it into the SmaI site of a binary vector pGK-X (Qin et al., The Plant Cell 20:1693-1707 (2008)) and the nucleotide sequence was then confirmed. This vector was introduced into a soil bacterium strain (Agrobacterium tumefaciens strain GV3101 (C58C1Rifr) pMP90 (Gmr) (Konez, C., and Schell, J. 1986. Mol. Gen. Genet. 204: 383-396)) by electroporation. Electroporation was performed under the following conditions. Agrobacterium and DNA of the prepared construct were placed in a 0.1-cm cuvette and a pulse was delivered once (voltage: 2.2 (kV)) using a Micro Pulser™ (Bio-Rad Laboratories, Inc.). The transformed Agrobacterium was then cultured on agar medium to which kanamycin (30 mg/l), rifampicin (50 mg/l), gentamicin (25 mg/l), and tetracycline (5 mg/l) had been added for 2 days. The obtained colonies were cultured in a similar liquid medium and used in the floral-dip method described below.

Transformation of Arabidopsis thaliana (ecotype Col-0) was performed by the floral-dip method. The transformed Agrobacterium cells were cultured in 1 liter of a YEP medium containing antibiotics (kanamycin (Km): 30 mg/l; gentamicin (Gm): 25 mg/l; rifampicin (Rif): 50 mg/l; and tetracycline: 5 mg/l) until OD600 reached 1. Subsequently, the cells were recovered from the culture and suspended in 1 liter of an infection medium to obtain a cell suspension. The composition of this infection medium is as follows: ½×MS salt, 0.112× Gamborg B5 vitamin, 5% sucrose, 10 μg/L benzyladenine and 0.02% Silwet L-77.

Arabidopsis thaliana plants that had been grown for 14 days were soaked in the cell suspension for 1 minute for infection and further grown for seed maturation. Collected seeds were sterilized in a solution containing 1% sodium hypochlorite and 0.02% TritonX-100 for 10 minutes, rinsed with sterile water three times, and sown on a sterilized kanamycin selection medium. The composition of the kanamycin selection medium is as follows: 1×MS salt, 3% sucrose, 0.05% MES (pH=5.7), 1× Gamborg B5 vitamin, 30 μg/L kanamycin, 200 μg/L Claforan® and 0.8% agar.

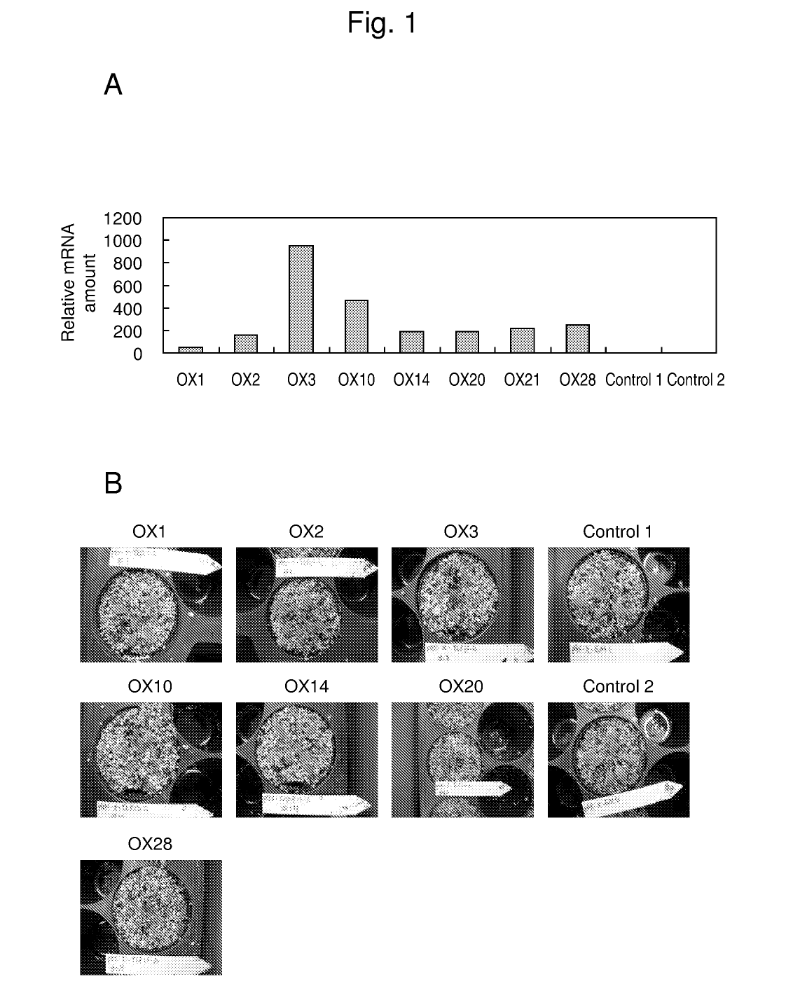

Transformed plants OX1, OX2, OX3, OX10, OX14, OX20, OX21 and OX28 were selected as kanamycin resistance plants from among plants grown from the sown seeds. Overexpression of the TCP13 gene in T2 transformants of the selected OX plants was confirmed (FIGS. 1A and 1B). The transformants were used in the stress resistance test in the Examples described below.

FIG. 1A shows TCP13 overexpression in transformed strains OX1, OX2, OX3, OX10, OX14, OX20, OX21 and OX28. The TCP13 expression levels were particularly high in OX3 and OX10. On the other hand, TCP13 expression was not detected in controls (controls 1 and 2). FIG. 1B shows the results of observation of the growth status of the transformants. The growth of the transformants was satisfactory, although the transformants tended to be smaller than the controls on the whole.

### EXAMPLE 2

### Salt Stress Resistance Test on TCP13-Overexpressing Plants

The strains OX3, OX10, OX14 and OX20 from among the transformed strains produced in Example 1 and control plants into which an empty pGK-X vector had been introduced were separately sown on a square plate in which nylon mesh was placed on an MS agar medium containing 1% sucrose and 50 mg/l kanamycin, and they were grown at 22° C. under long-day conditions comprising 16 hours in the light and 8 hours in the dark for 7 days. Thereafter, each plant was transferred together with nylon mesh onto an MS agar medium containing 225 mM NaCl and 1% sucrose and grown under similar long-day conditions for 12 days.

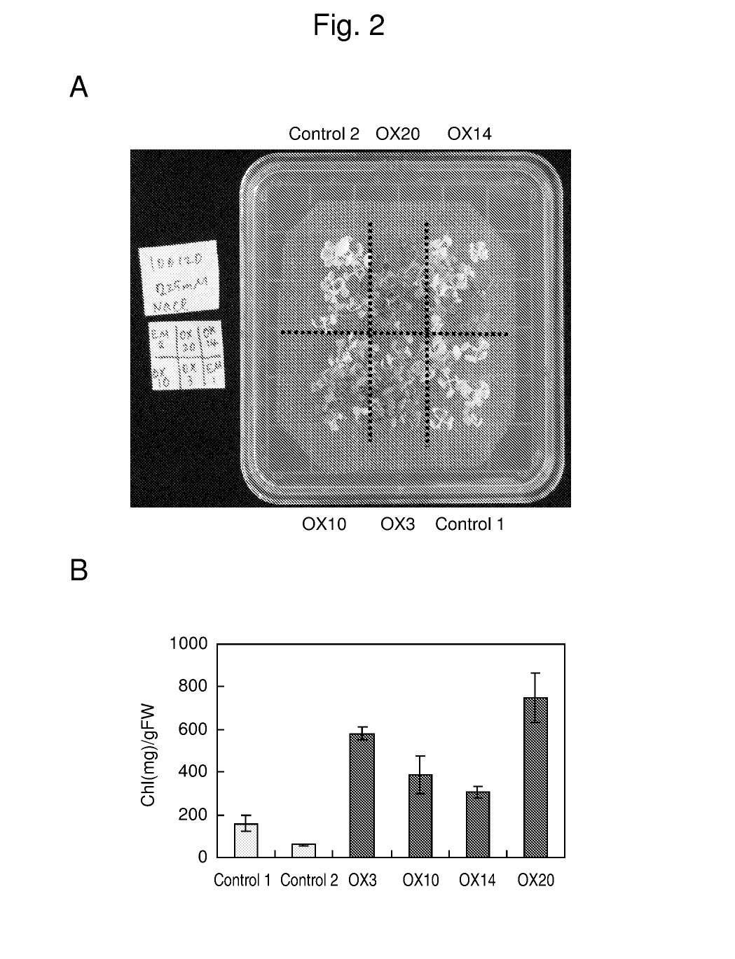

As is apparent from FIG. 2A, all of the transformants of the present invention survived under high salinity, while the controls withered and died.

The aerial part of each plant was collected, measured, and soaked in dimethylformamide in the dark for 24 hours four times, followed by chlorophyll fluorescence measurement. FIG. 2B shows that the chlorophyll content of each of the strains OX3, OX10, OX14 and OX20, and especially OX3 and OX20, was higher than that of each control, consistent with the results shown in FIG. 2A.

### EXAMPLE 3

### Drought Stress Resistance Test on TCP13-Overexpressing Plants

The strains OX3 and OX28 from among the transformed strains produced in Example 1 and the control plants into which an empty pGK-X vector had been introduced were separately sown on an MS agar medium containing 3% sucrose and 50 mg/l kanamycin and grown at 22° C. under long-day conditions comprising 16 hours in the light and 8 hours in the dark for 14 days. Each plant was then transferred to culture soil (Professional Baiyoudo “Sashiki/Sashime You” (professional culture soil “for cutting/herbaceous cutting”), Dio Chemicals, Ltd.) and grown under long-day conditions for 1 week. Then, water supply was stopped. Water supply was resumed 13 days after the stop of water supply, and the survival rate was calculated.

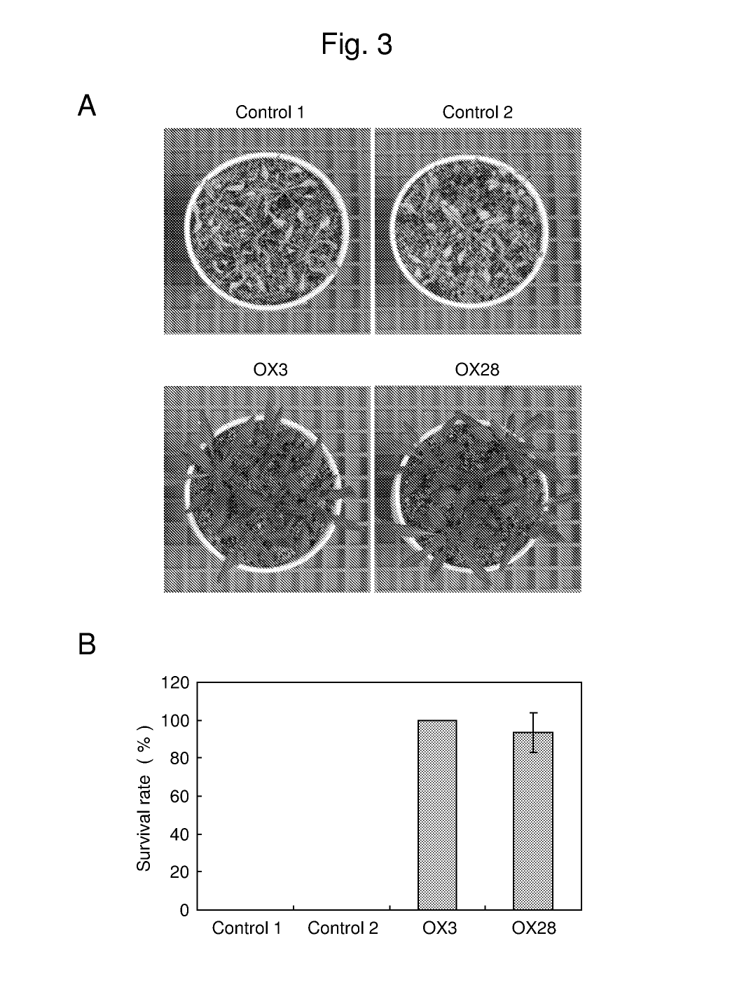

FIG. 3 shows appearances of transformed plants for which water supply had been resumed 13 days after stop of water supply and which were then grown under usual growth conditions for 7 days. OX3 and OX28 had high survival rates, while the control plants withered and died.

### EXAMPLE 4

### Production of TCP5-Overexpressing Plants and TCP17-Overexpressing Plants, and Salt Stress Resistance Test on these Plants

Arabidopsis thaliana was transformed as in the case of Example 1, except that PCR reaction was performed using TCP5F (ATGAGATCAGGAGAATGTGATG: SEQ ID NO: 105) and TCP5R (TCAAGAATCTGATTCATTATCGC: SEQ ID NO: 106) as PCR primers for TCP5 gene amplification, or TCP17F (ATGGGAATAAAAAAAGAAGATC: SEQ ID NO: 107) and TCP17R (CTACTCGATATGGTCTGGTTGTG: SEQ ID NO: 108) as PCR primers for TCP17 gene amplification. Accordingly, the strains 5OX-10, 5OX-16, 5OX-17, and 5OX-23 were obtained by transformation with the TCP5 gene and the strains 17OX-3, 17OX-9, 17OX-14 and 17OX-15 were obtained by transformation with the TCP17 gene.

The salt stress test and chlorophyll fluorescence measurement were carried out as in the case of Example 2 using the produced transformed strains.

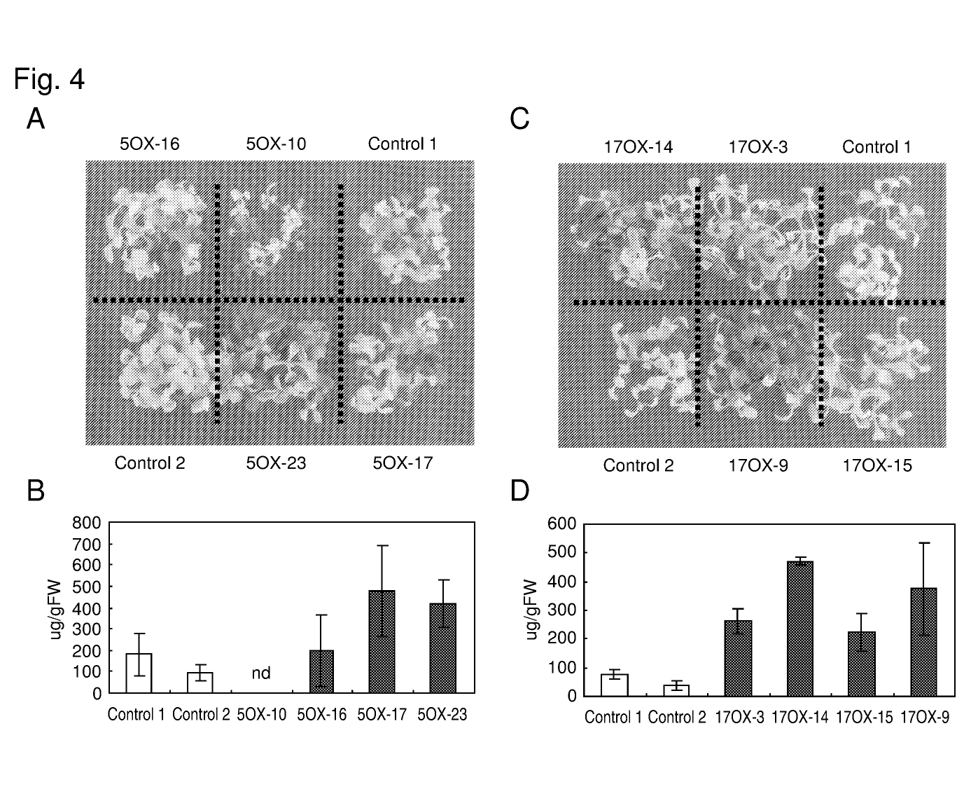

As shown in FIG. 4A, TCP5-overexpressing plants, except the 5OX-10 strain, were superior to the controls in terms of growth status. In addition, as shown in FIG. 4B, the chlorophyll content of each of the TCP5-overexpressing plants, except the 5OX-10 strain, was higher than that of each control, consistent with the results shown in FIG. 4A.

In addition, as shown in FIG. 4C, the TCP17-overexpressing plants were superior to the controls in terms of growth status. Also as shown in FIG. 4D, the chlorophyll content of each of the TCP17-overexpressing plant, and especially the strains 17OX-14 and 17OX-9, was higher than that of each control, consistent with the results shown in FIG. 4C.

The amino acid sequence and the nucleotide sequence of Arabidopsis thaliana TCP13 used in the above Examples are shown below (AT3G02150.2).

<Amino acid sequence of TCP13
(Arabidopsis thaliana)
(SEQ ID NO: 1)>
MNIVSWKDANDEVAGGATTRREREVKEDQEETEVRATSGKTVIKKQPTSI
SSSSSSWMKSKDPRIVRVSRAFGGKDRHSKVCTLRGLRDRRVRLSVPTAI
QLYDLQERLGVDQPSKAVDWLLDAAKEEIDELPPLPISPENFSIFNHHQS
FLNLGQRPGQDPTQLGFKINGCVQKSTTTSREENDREKGENDVVYTNNHH
VGSYGTYHNLEHHHHHHQHLSLQADYHSHQLHSLVPFPSQILVCPMTTSP
TTTTIQSLFPSSSSAGSGTMETLDPRQMVSHFQMPLMGNSSSSSSQNIST
LYSLLHGSSSNNGGRDIDNRMSSVQFNRTNSTTTANMSRHLGSERCTSRG
SDHHM
<Nucleotide sequence of TCP13
(Arabidopsis thaliana)
(SEQ ID NO: 2)>
ATGAATATCGTCTCTTGGAAAGATGCAAACGACGAAGTTGCAGGCGGCGC
TACGACAAGACGTGAAAGAGAAGTAAAAGAGGATCAAGAAGAAACCGAAG
TCAGAGCCACCAGTGGCAAAACCGTAATTAAAAAGCAGCCTACATCGATC
TCTTCTTCTTCTTCTTCGTGGATGAAATCCAAGGATCCGAGGATTGTTAG
GGTTTCACGCGCCTTTGGAGGCAAAGACCGTCACAGCAAAGTGTGTACGT
TACGTGGACTACGTGACAGACGCGTGAGATTATCAGTCCCAACGGCTATT
CAGCTCTACGATCTTCAAGAACGGCTCGGTGTTGACCAGCCTAGCAAAGC
CGTTGACTGGTTGCTTGATGCAGCTAAAGAGGAGATCGACGAGCTACCTC
CGTTACCTATCTCGCCGGAAAATTTCAGCATCTTCAACCATCATCAGTCC
TTCTTGAATCTTGGTCAACGGCCCGGTCAAGATCCGACCCAACTCGGGTT
TAAAATCAATGGATGTGTACAAAAGTCTACTACTACTAGCCGCGAAGAAA
ACGATAGAGAGAAAGGAGAAAACGATGTCGTTTACACAAACAATCATCAT
GTTGGGTCTTATGGAACTTATCACAACCTGGAACATCATCATCATCATCA
CCAACATTTGAGTTTACAGGCAGATTATCATAGTCATCAACTACATAGTC
TTGTCCCATTTCCATCACAAATTTTGGTATGTCCAATGACGACATCACCA
ACAACTACAACTATACAATCTTTGTTTCCATCATCATCGTCAGCTGGTTC
AGGGACTATGGAGACATTAGATCCGAGGCAAATGGTAAGCCATTTTCAAA
TGCCATTAATGGGTAATTCTTCATCTTCGTCTTCCCAAAACATTTCGACA
TTATATTCGTTGTTACATGGTAGTAGTAGCAACAATGGTGGTAGAGACAT
TGATAATCGGATGTCGTCGGTCCAATTCAACCGAACTAATAGCACTACAA
CGGCTAACATGTCGAGGCATCTAGGCTCGGAGCGTTGTACAAGTAGAGGA
AGTGATCACCATATGTGA

In addition, the amino acid sequences and the nucleotide sequences of Arabidopsis thaliana TCP5 (AT5G60970) and TCP17 (AT5G08070) used in the above Examples are shown below.

<Amino acid sequence of TCP5
(Arabidopsis thaliana)
(SEQ ID NO: 5)>
MRSGECDEEEIQAKQERDQNQNHQVNLNHMLQQQQPSSVSSSRQWTSAFR
NPRIVRVSRTFGGKDRHSKVCTVRGLRDRRIRLSVPTAIQLYDLQDRLGL
SQPSKVIDWLLEAAKDDVDKLPPLQFPHGFNQMYPNLIFGNSGFGESPSS
TTSTTFPGTNLGFLENWDLGGSSRTRARLTDTTTTQRESFDLDKGKWIKN
DENSNQDHQGFNTNHQQQFPLTNPYNNTSAYYNLGHLQQSLDQSGNNVTV
AISNVAANNNNNLNLHPPSSSAGDGSQLFFGPTPPAMSSLFPTYPSFLGA
SHHHHVVDGAGHLQLFSSNSNTASQQHMMPGNTSLIRPFHHLMSSNHDTD
HHSSDNESDS*
<Nucleotide sequence of TCP5
(Arabidopsis thaliana)
(SEQ ID NO: 6)>
ATGAGATCAGGAGAATGTGATGAAGAGGAGATTCAAGCAAAGCAAGAAAG
AGATCAAAATCAAAATCATCAAGTAAACTTAAACCACATGTTGCAACAAC
AACAGCCGAGTTCGGTATCATCTTCAAGGCAATGGACTTCAGCTTTTAGG
AATCCAAGAATCGTTCGAGTCTCAAGAACATTCGGTGGCAAAGACAGACA
CAGCAAAGTATGTACAGTCCGTGGTCTTCGAGACCGGAGGATAAGGTTGT
CCGTACCTACAGCTATTCAACTCTACGACCTTCAAGATCGATTAGGGCTG
AGTCAGCCAAGCAAAGTCATTGATTGGTTACTCGAAGCAGCAAAAGATGA
CGTAGACAAGCTACCTCCTCTACAATTCCCACATGGATTTAACCAGATGT
ATCCAAATCTCATCTTCGGAAACTCCGGGTTTGGAGAATCTCCATCTTCA
ACTACATCAACAACGTTTCCAGGAACCAATCTCGGGTTCTTGGAAAATTG
GGATCTTGGTGGTTCTTCAAGAACAAGAGCAAGATTAACCGATACAACTA
CGACCCAAAGAGAAAGTTTTGATCTTGATAAAGGAAAATGGATCAAAAAC
GACGAGAATAGTAATCAAGATCATCAAGGGTTTAACACCAATCATCAACA
ACAATTTCCTCTGACCAATCCGTACAACAACACTTCAGCTTATTACAACC
TTGGACATCTTCAACAATCGTTAGACCAATCTGGTAATAACGTTACTGTC
GCAATATCTAATGTTGCTGCTAATAATAACAATAATCTCAATTTGCATCC
TCCTTCCTCGTCTGCCGGAGATGGATCTCAGCTTTTTTTCGGTCCTACTC
CTCCGGCAATGAGCTCTCTATTCCCGACATACCCTTCGTTTCTTGGAGCT
TCTCATCATCATCATGTCGTCGATGGAGCCGGTCATCTTCAGCTCTTTAG
CTCGAATTCAAATACCGCATCGCAGCAACACATGATGCCGGGTAATACGA
GTTTGATTAGACCATTTCATCATTTGATGAGCTCGAATCATGATACGGAT
CATCATAGTAGCGATAATGAATCAGATTCTTGA
<Amino acid sequence of TCP17
(Arabidopsis thaliana)
(SEQ ID NO: 7)>
MGIKKEDQKSSLSLLTQRWNNPRIVRVSRAFGGKDRHSKVCTVRGLRDRR
IRLSVMTAIQVYDLQERLGLSQPSKVIDWLLEVAKNDVDLLPPLQFPPGF
HQLNPNLTGLGESFPGVFDLGRTQREALDLEKRKWVNLDHVFDHIDHHNH
FSNSIQSNKLYFPTITSSSSSYHYNLGHLQQSLLDQSGNVTVAFSNNYNN
NNLNPPAAETMSSLFPTRYPSFLGGGQLQLFSSTSSQPDHIE*
<Nucleotide sequence of TCP17
(Arabidopsis thaliana)
(SEQ ID NO: 8)>
ATGGGAATAAAAAAAGAAGATCAGAAAAGTTCCTTGTCCTTGTTGACGCA
ACGATGGAATAATCCGAGGATCGTGCGAGTCTCCCGAGCATTTGGAGGCA
AAGATAGACACAGCAAAGTGTGCACAGTTCGTGGTCTCCGAGACAGGAGG
ATAAGGTTGTCGGTGATGACAGCGATCCAAGTCTACGACCTTCAAGAACG
ATTAGGGCTGAGTCAGCCTAGCAAAGTCATTGATTGGCTCTTAGAAGTTG
CCAAAAACGACGTCGATTTGCTTCCTCCTTTGCAATTCCCACCTGGTTTT
CACCAACTTAACCCTAATCTCACCGGCCTTGGAGAATCTTTTCCCGGGGT
CTTCGATCTTGGAAGAACACAGAGAGAAGCTTTGGATCTTGAGAAAAGGA
AATGGGTTAATCTTGATCATGTGTTTGATCACATCGATCATCACAACCAT
TTCTCGAATTCAATCCAGAGCAACAAGCTTTATTTCCCTACAATTACTTC
GTCTAGCAGCTCTTATCACTACAACCTTGGACATCTCCAACAGTCACTAC
TAGACCAATCTGGTAACGTCACTGTCGCATTTTCTAATAACTATAATAAT
AATAATCTCAATCCTCCGGCGGCGGAAACTATGAGTTCTCTATTCCCAAC
AAGGTACCCTTCGTTTCTCGGTGGTGGTCAACTTCAACTCTTTAGTAGCA
CAAGCTCACAACCAGACCATATCGAGTAG

All publications, patents, and patent applications cited herein are incorporated herein by reference in their entirety.

### INDUSTRIAL APPLICABILITY

According to the present invention, resistance to environmental stress such as drought stress or salt stress can be conferred on a plant. Thus, the present invention can be used in the fields of agriculture and forestry, in particular.

### SEQUENCE LISTING FREE TEXT

SEQ ID NO: 103 and 104: Primers
SEQ ID NO: 105 and 106: Primers
SEQ ID NO: 107 and 108: Primers

1.-9. (canceled)

10. A method of producing a transgenic plant having environmental stress resistance, comprising:
introducing an exogenous DNA encoding a transcription factor selected from the group consisting of transcription factors from Arabidopsis thaliana TCP13, TCP5 and TCP17, and homologous transcription factors thereof from other plants into a cell or tissue of a plant in an expressible manner; and
regenerating a plant from the cell or tissue.

11. The method according to claim 10, wherein the transcription factor TCP13 or the homologous transcription factor thereof comprises the following protein (a) or (b):
(a) a protein consisting of the amino acid sequence shown in SEQ ID NO: 1, 3, 13, 15, 17, 19, 21, 23, 33, 35, 37, 39, 43, 55, 57, 59, 61, 63,67, 69, 75, 77, 79, 95, 97, 99 or 101; or
(b) a protein consisting of an amino acid sequence having at least 70% identity to the amino acid sequence as recited in (a) and being capable of conferring environmental stress resistance on a plant.

12. The method according to claim 10, wherein the transcription factor TCP5 or TCP17, or the homologous transcription factor thereof comprises the following protein (c) or (d):
(c) a protein consisting of an amino acid sequence shown in SEQ ID NO: 5, 7, 9, 11, 13, 15, 17, 25, 27, 29, 31, 33, 35, 37, 39, 41, 45, 47, 49, 51, 53, 55, 57, 65, 67, 69, 71, 73, 75, 77, 79, 81, 83, 85, 87, 89, 91, 95, 97, 99 or 101; or
(d) a protein consisting of an amino acid sequence having at least 70% identity to the amino acid sequence as recited in (c) and being capable of conferring environmental stress resistance on a plant.

13. The method according to claim 10, wherein the DNA encoding the transcription factor TCP13 or the homologous transcription factor thereof is any of the following DNAs (e) to (g):
(e) DNA consisting of the nucleotide sequence shown in SEQ ID NO: 2, 4, 14, 16, 18, 20, 22, 24, 34, 36, 38,40,44, 56,58,60,62,64,68,70,76, 78, 80, 96,98, 100 or 102;
(f) DNA consisting of a nucleotide sequence having at least 70% identity to the nucleotide sequence as recited in (e) and encoding a protein capable of conferring environmental stress resistance on a plant;
(g) DNA that hybridizes under stringent conditions to DNA consisting of a nucleotide sequence complementary to DNA consisting of the nucleotide sequence as recited in (e) and encodes a protein capable of conferring environmental stress resistance on a plant.

14. The method according to claim 10, wherein the DNA encoding the transcription factor TCPS or TCP17, or the homologous transcription factor thereof is any of the following DNAs (h) to (j):
(h) DNA consisting of the nucleotide sequence shown in SEQ ID NO: 6, 8, 10, 12, 14, 16, 18,26, 28, 30, 32,34, 36, 38, 40, 42,46, 48, 50, 52, 54, 56, 58, 66, 68, 70, 72, 74, 76, 78, 80, 82, 84, 86, 88, 90, 92, 94, 96, 98, 100 or 102;
(i) DNA consisting of a nucleotide sequence having at least 70% identity to the nucleotide sequence as recited in (h) and encoding a protein capable of conferring environmental stress resistance on a plant;
(j) DNA that hybridizes under stringent conditions to DNA consisting of a nucleotide sequence complementary to DNA consisting of the nucleotide sequence as recited in (h) and encodes a protein capable of conferring environmental stress resistance on a plant.

15. The method according to claim 10, wherein the environmental stress resistance is salt stress resistance or drought stress resistance.

16. A method of conferring environmental stress resistance on a plant, comprising:
introducing an exogenous DNA encoding a transcription factor selected from the group consisting of transcription factors from Arabidopsis thaliana TCP13, TCPS and TCP17, and homologous transcription factors thereof from other plants into a cell or tissue of a plant in an expressible manner; and
regenerating a plant from the cell or tissue.

17. The method according to claim 16, wherein the transcription factor TCP13 or the homologous transcription factor thereof comprises the following protein (a) or (b):
(a) a protein consisting of the amino acid sequence shown in SEQ ID NO: 1, 3, 13, 15, 17, 19,21,23, 33, 35, 37, 39,43, 55, 57, 59, 61, 63, 67, 69, 75, 77, 79, 95, 97, 99 or 101; or
(b) a protein consisting of an amino acid sequence having at least 70% identity to the amino acid sequence as recited in (a) and being capable of conferring environmental stress resistance on a plant.

18. The method according to claim 16, wherein the transcription factor TCPS or TCP17, or the homologous transcription factor thereof comprises the following protein (c) or (d):
(c) a protein consisting of an amino acid sequence shown in SEQ ID NO: 5, 7, 9, 11, 13, 15, 17, 25, 27, 29, 31, 33, 35, 37, 39, 41, 45, 47, 49, 51, 53, 55, 57, 65, 67, 69, 71, 73, 75, 77, 79, 81, 83, 85, 87, 89, 91, 95, 97, 99 or 101; or
(d) a protein consisting of an amino acid sequence having at least 70% identity to the amino acid sequence as recited in (c) and being capable of conferring environmental stress resistance on a plant.

19. The method according to claim 16, wherein the DNA encoding the transcription factor TCP13 or the hoinologous transcription factor thereof is any of the following DNAs (e) to (g):
(e) DNA consisting of the nucleotide sequence shown in SEQ ID NO: 2, 4, 14, 16, 18, 20, 22, 24, 34, 36, 38, 40, 44, 56, 58, 60, 62, 64, 68, 70, 76, 78, 80, 96, 98, 100 or 102;
(f) DNA consisting of a nucleotide sequence having at least 70% identity to the nucleotide sequence as recited in (e) and encoding a protein capable of conferring environmental stress resistance on a plant;
(g) DNA that hybridizes under stringent conditions to DNA consisting of a nucleotide sequence complementary to DNA consisting of the nucleotide sequence as recited in (e) and encodes a protein capable of conferring environmental stress resistance on a plant.

20. The method according to claim 16, wherein the DNA encoding the transcription factor TCP5 or TCP17, or the homologous transcription factor thereof is any of the following DNAs (h) to (j):
(h) DNA consisting of the nucleotide sequence shown in SEQ ID NO: 6, 8, 10, 12, 14, 16, 18,26, 28, 30, 32,34, 36, 38,40,42,46,48, 50, 52, 54, 56, 58, 66, 68, 70, 72, 74, 76, 78, 80, 82, 84, 86, 88,90, 92, 94, 96, 98, 100 or 102;
(i) DNA consisting of a nucleotide sequence having at least 70% identity to the nucleotide sequence as recited in (h) and encoding a protein capable of conferring environmental stress resistance on a plant;
(j) DNA that hybridizes under stringent conditions to DNA consisting of a nucleotide sequence complementary to DNA consisting of the nucleotide sequence as recited in (h) and encodes a protein capable of conferring environmental stress resistance on a plant.

21. The method according to claim 16, wherein the environmental stress resistance is salt stress resistance or drought stress resistance.
